(19) United States
(12) Patent Application Publication
(10) Pub. No.: US 2020/0261421 A1
(43) Pub. Date: Aug. 20, 2020

(54) HETEROCYCLIC COMPOUNDS AS HIV PROTEASE INHIBITORS

(71) Applicant: Merck Sharp & Dohme Corp., Rahway, NJ (US)

(72) Inventors: Brian McKittrick, New Vernon, NJ (US); John P. Caldwell, Ringwood, NJ (US); John A. McCauley, Maple Glen, PA (US); Henry Vaccaro, South Plainfield, NJ (US); Tin-Yau Chan, Edison, NJ (US); Hyunjin Kim, Livingston, NJ (US); Elizabeth Smith, Verona, NJ (US); Liwu Hong, East Brunswick, NJ (US); Tanweer Khan, Bridgewater, NJ (US); Shihong Ying, Carteret, NJ (US); Hongwu Wang, Westfield, NJ (US)

(73) Assignee: Merck Sharp & Dohme Corp., Rahway, NJ (US)

(21) Appl. No.: 16/461,952

(22) PCT Filed: Dec. 19, 2017
(86) PCT No.: PCT/US17/67160

§ 371 Date: May 17, 2019

Related U.S. Application Data
(60) Provisional application No. 62/437,928, filed on Dec. 22, 2016.

(51) Int. Cl. A61K 31/4168; A61K 45/06; A61K 31/497; C07D 233/72; C07D 403/12; C07D 401/12; A61P 31/18

(52) U.S. Cl. CPC A61K 31/4168; A61K 45/06; A61K 31/497; A61P 31/18; C07D 403/12; C07D 401/12; C07D 233/72

(57) ABSTRACT

The present invention is directed to compounds of Formula I, pharmaceutical compositions comprising the same, and their use in the inhibition of HIV protease, the inhibition of HIV replication, the prophylaxis of infection by HIV, the treatment of infection by HIV, and the prophylaxis, treatment, and delay in the onset or progression of AIDS.

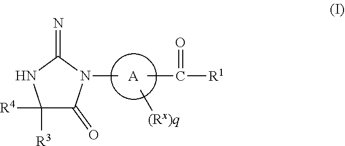

### BACKGROUND OF THE INVENTION

A retrovirus designated human immunodeficiency virus (HIV), particularly the strains known as HIV type-1 (HIV-1) virus and type-2 (HIV-2) virus, is the etiological agent of acquired immunodeficiency syndrome (AIDS). AIDS is a disease characterized by the destruction of the immune system, particularly of CD4 T-cells, with attendant susceptibility to opportunistic infections, and its precursor AIDS-related complex (“ARC”), a syndrome characterized by symptoms such as persistent generalized lymphadenopathy, fever and weight loss. This virus was previously known as LAV, HTLV-III, or ARV. A common feature of retrovirus replication is the extensive post-translational processing of precursor polyproteins by a virally encoded protease to generate mature viral proteins required for virus assembly and function. Inhibition of this processing prevents the production of normally infectious virus. For example, Kohl et al., Proc. Nat'l Acad. Sci. 1988, 85: 4686, demonstrated that genetic inactivation of the HIV encoded protease resulted in the production of immature, non-infectious virus particles. These results indicated that inhibition of the HIV protease represents a viable method for the treatment of AIDS and the prevention or treatment of infection by HIV.

Nucleotide sequencing of HIV shows the presence of a pol gene in one open reading frame [Ratner et al., Nature 1985, 313: 277]. Amino acid sequence homology provides evidence that the pol sequence encodes reverse transcriptase, an endonuclease, HIV protease and gag, which encodes the core proteins of the virion (Toh et al., EMBO 11985, 4: 1267; Power et al., Science 1986, 231: 1567; Pearl et al., Nature 1987, 329: 351].

Several HIV protease inhibitors are presently approved for clinical use in the treatment of AIDS and HIV infection, including indinavir (see U.S. Pat. No. 5,413,999), amprenavir (U.S. Pat. No. 5,585,397), saquinavir (U.S. Pat. No. 5,196,438), ritonavir (U.S. Pat. No. 5,484,801) and nelfinavir (U.S. Pat. No. 5,484,926). Each of these protease inhibitors is a peptide-derived peptidomimetic, competitive inhibitor of the viral protease which prevents cleavage of the HIV gag-pol polyprotein precursor. Tipranavir (U.S. Pat. No. 5,852,195) is a non-peptide peptidomimetic protease inhibitor also approved for use in treating HIV infection. The protease inhibitors are administered in combination with at least one and typically at least two other HIV antiviral agents, particularly nucleoside reverse transcriptase inhibitors such as zidovudine (AZT) and lamivudine (3TC) and/or non-nucleoside reverse transcriptase inhibitors such as efavirenz and nevirapine. Indinavir, for example, has been found to be highly effective in reducing HIV viral loads and increasing CD4 cell counts in HIV-infected patients, when used in combination with nucleoside reverse transcriptase inhibitors. See, for example, Hammer et al., New England J. Med. 1997, 337: 725-733 and Gulick et al., New England J. Med. 1997, 337: 734-739.

There is a continuing need for new compounds which are capable of treating infectious diseases, in particular, for inhibiting HIV protease and suitable for use in the treatment or prophylaxis of infection by HIV and/or for the treatment or prophylaxis or delay in the onset or progression of AIDS.

### SUMMARY OF THE INVENTION

The present invention is directed to heterocycylic compounds, pharmaceutical compositions comprising the same, and their use in the inhibition of HIV protease, the inhibition of HIV replication, the prophylaxis of infection by HIV, the treatment of infection by HIV, and the prophylaxis, treatment, and delay in the onset or progression of AIDS.

### DETAILED DESCRIPTION OF THE INVENTION

The invention encompasses compounds of structural formula I

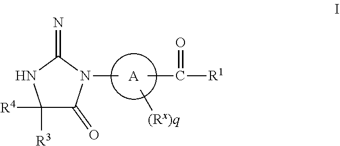

or a pharmaceutically acceptable salt thereof, wherein:
A is selected from the group consisting of (CHR²)ₚC₆₋₁₀aryl and (CHR²)ₚC₃₋₁₁heteroaryl;
R is selected from the group consisting of hydrogen and C₁₋₆alkyl;
R^{X} is selected from the group consisting of C₁₋₆alkyl, C₁₋₃haloalkyl, halogen, SO₂C₁₋₆alkyl, and OC₁₋₆alkyl;
R¹ is selected from the group consisting of —N(R)₂, NR(CHR)ₙC₄₋₁₂heterocyclyl, NR(CHR)ₙC₆₋₁₀aryl, and —N(R)CH₂C₁₋₃haloalkyl, said aryl, and heterocyclyl optionally substituted with 1 to 3 groups of R^{d};
R² is selected from the group consisting of hydrogen, C₁₋₆alkyl, C₁₋₃haloalkyl, halogen, SO₂C₁₋₆alkyl, (CH₂)ₙOC₁₋₆alkyl, (CH₂)ₙC₃₋₆cycloalkyl, (CHR)ₙC₆₋₁₀aryl, (CHR)ₙC₅₋₁₀heteroaryl said alkyl, aryl, and heteroaryl optionally substituted with 1 to 3 groups of R^{d};
R³ and R⁴ are independently selected from the group consisting of C₁₋₆alkyl, (CH₂)ₙC₁₋₃haloalkyl, (CR₂)ₙC₃₋₆cycloalkyl, (CH₂)ₙC₆₋₁₀aryl, (CH₂)ₙC₅₋₁₀heterocyclyl; said alkyl, aryl, and heterocyclyl optionally substituted with 1 to 3 groups of R^{d};
R⁵ is selected from the group consisting of hydrogen, C₁₋₆alkyl, C(O)OR, C₃₋₆cycloalkyl, SO₂R, O(CH₂)ₙC₆₋₁₀aryl, and (CH₂)ₙC₆₋₁₀aryl;
R^{d} is selected from the group consisting of C₁₋₆alkyl, C₁₋₃haloalkyl, OC₁₋₃haloalkyl, OC₁₋₆alkyl, CN, ═O, SO₂R, C(O)NR₂, C₅₋₁₀heteroaryl, C₆₋₁₀aryl, and halogen, said heteroaryl, alkyl and aryl optionally substituted with 1 to 3 groups of halogen and CN;
n is 0, 1, 2, 3, or 4;
p is 0 or 1; and
q is 0 or 1.

An embodiment of the invention of formula I is realized when A is (CHR²)ₚC₆₋₁₃aryl. An embodiment of the invention of formula I is realized when A is aryl and p=1. A subembodiment of this aspect of the invention is realized when A is an aryl selected from the group consisting of phenyl, tetrahydronaphthalenyl, dihydroindenyl, and tetrahydrobenzoannulenyl. Another subembodiment of this aspect of the invention is realized when A is phenyl and p=1. Another subembodiment of this aspect of the invention is realized when A is tetrahydronaphthalenyl and p=0. Still another subembodiment of this aspect of the invention is realized when A is dihydroindenyl and p=0 . Yet another subembodiment of this aspect of the invention is realized when A is tetrahydrobenzoannulenyl and p=0.

Another embodiment of the invention of formula I is realized when A is (CHR²)ₚC₅₋₁₁ 11heteroaryl. Another embodiment of the invention of formula I is realized when A is (CHR²)ₚC₅₋₁₁heteroaryl and p=1. Another embodiment of the invention of formula I is realized when A is (CHR²)ₚC₉₋₁₁heteroaryl and p=0. A subembodiment of this aspect of the invention is realized when A is a heteroaryl selected from the group consisting of pyridyl, thiazolyl, thiophenyl, dihydrochromenyl, and dihydrothiochromenyl. Another subembodiment of this aspect of the invention is realized when A is pyridyl. Another subembodiment of this aspect of the invention is realized when A is thiazolyl. Still another subembodiment of this aspect of the invention is realized when A is thiophenyl. Yet another subembodiment of this aspect of the invention is realized when A is dihydrochromenyl. Yet another subembodiment of this aspect of the invention is realized when A is dihydrothiochromenyl. Another embodiment of the invention of formula I is realized when A is is C₅₋₁₁heteroaryl which is pyridyl and p=1.

Another embodiment of the invention of formula I is realized when R¹ is —N(R)₂. A subembodiment of this aspect of the invention is realized when R is selected from the group consisting of CH₃, CH₂CH₃, and (CH₂)ₙCH(CH₃)₂.

Another embodiment of the invention of formula I is realized when R¹ is —NR(CHR)ₙC₄₋₁₂heterocyclyl, said heterocyclyl optionally substituted with 1 to 3 groups of R^{d}. A subembodiment of this aspect of the invention is realized when the heterocyclyl is selected from the group consisting of optionally substituted pyridyl, pyrimidinyl, and pyrazinyl.

Another embodiment of the invention of formula I is realized when R¹ is —NR(CHR)ₙC₆₋₁₀aryl, said aryl optionally substituted with 1 to 3 groups of R^{d}. A subembodiment of this aspect of the invention is realized when aryl is selected from optionally substituted phenyl.

Another embodiment of the invention of formula I is realized when R¹ is —N(R)CH₂C₁₋₃haloalkyl. A subembodiment of this aspect of the invention is realized when the haloalkyl is CF₃.

An embodiment of the invention of formula I is realized when R² is hydrogen.

Another embodiment of the invention of formula I is realized when R² is optionally substituted C₁₋₆alkyl or (CH₂)ₙOC₁₋₆alkyl. A subembodiment of this aspect of the invention is realized when R² is optionally substituted C₁₋₆alkyl. Another subembodiment of this aspect of the invention is realized when R² is optionally substituted (CH₂)ₙOC₁₋₆alkyl. Another subembodiment of this aspect of the invention is realized when the alkyl is selected from the group consisting of CH₃, CH₂CH₃, (CH₂)ₙCH(CH₃)₂, and (CH₂)ₙOCH(CH₃)₂.

Another embodiment of the invention of formula I is realized when R₂ is optionally substituted (CH₂)ₙC₃₋₆cycloalkyl. A subembodiment of this aspect of the invention is realized when the cycloalkyl is selected from the group consisting of cyclopropyl, cyclobutyl and cyclopentyl.

Another embodiment of the invention of formula I is realized when R² is optionally substituted (CHR)ₙC₆₋₁₀aryl. A subembodiment of this aspect of the invention is realized when the aryl is phenyl.

Another embodiment of the invention of formula I is realized when R² is optionally substituted (CHR)ₙC₅₋₁₀heteroaryl. A subembodiment of this aspect of the invention is realized when the heteroaryl is pyrrollidinone.

An embodiment of the invention of formula I is realized when one of R³ and R⁴ is optionally substituted C₁₋₆alkyl, (CH₂)ₙC₁₋₃haloalkyl, or (CR₂)ₙC₃₋₆cycloalkyl and the other is (CH₂)ₙC₆₋₁₀aryl, (CH₂)ₙC₅₋₁₀heterocyclyl; said alkyl, aryl, and heterocyclyl optionally substituted with 1 to 3 groups of R^{d}.

Another embodiment of the invention of formula I is realized when R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}. A subembodiment of this aspect of the invention is realized when R³ and R⁴ are independently selected from the group consisting of isobutyl, isopentyl, (CH₂)ₙCF₃, (CH₂)ₙcyclopropyl, and phenyl, said isobutyl, isopentyl and phenyl optionally substituted with 1 to 3 groups of R^{d}. Another subembodiment of this aspect of the invention is realized when one of R³ and R⁴ is optionally substituted phenyl. Another subembodiment of this aspect of the invention is realized when both of R³ and R⁴ are optionally substituted phenyl. Another subembodiment of this aspect of the invention is realized when R⁴ is optionally substituted phenyl. Still another subembodiment of this aspect of the invention is realized when R⁴ is optionally substituted phenyl and R³ is selected from the group consisting of isobutyl, isopentyl, (CH₂)ₙCF₃, (CH₂)ₙcyclopropyl, phenyl, said isobutyl, isopentyl, cyclopropyl and phenyl optionally substituted with 1 to 3 groups of R^{d}. Still another subembodiment of this aspect of the invention is realized when R³ is optionally substituted phenyl and R⁴ is selected from the group consisting of isobutyl, isopentyl, (CH₂)ₙCF₃, (CH₂)ₙcyclopropyl, phenyl, said isobutyl, isopentyl, cyclopropyl and phenyl optionally substituted with 1 to 3 groups of R^{d}.

Another embodiment of the invention of formula I is realized when R^{d} is selected from the group consisting of (CH₂)ₙCH₃, CF₃, fluoro, chloro, bromo, CN, CO)NH₂, C(O)N(CH₃)₂, phenyl, pyridyl and furanyl, said phenyl, pyridyl and furanyl optionally substituted with 1 to 3 groups of halogen and CN.

Another embodiment of the invention of formula I is realized when R^{d} is present on R¹ and is selected from the group consisting of (CH₂)ₙCH₃, CF₃, fluoro, chloro, and bromo.

Another embodiment of the invention of formula I is realized when R^{X} is selected from the group consisting of CH₃, OCH₃, CF₃, SO₂CH₃, fluoro, and chloro.

Another embodiment of the invention of formula I is realized when p is 0 resulting in a bond as the linking group. Another embodiment of the invention of formula I is realized when p is 1.

Still another embodiment of the invention of formula I is realized when q is 0 which means the R^{X} group is not present.

Another embodiment of the invention of formula I is realized when q is 1.

Yet another embodiment of the invention of formula I is realized when n is 0. Another embodiment of the invention of formula I is realized when n is 1. Another embodiment of the invention of formula I is realized when n is 2. Another embodiment of the invention of formula I is realized when n is 3. Another embodiment of the invention of formula I is realized when n is 4.

In another embodiment of this invention are compounds of formula I having structural formula II, or the pharmaceutically acceptable salts thereof,

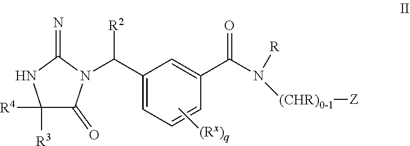

wherein R², R³, R⁴, R^{a} and R^{X} are as previously described and Z is selected from the group consisting of R, optionally substituted C₆₋₁₀aryl and optionally substituted C₅₋₁₀heteroaryl. A subembodiment of the invention of formula II is realized when Z is selected from the group consisting of unsubstituted or substituted R, phenyl, pyridyl, pyrimidinyl, and pyrazinyl. A subembodiment of the invention of formula II is realized when Z is substituted with 1 to 3 groups of R^{d} selected from the group consisting of (CH₂)ₙCH₃, CF₃, fluoro, chloro, and bromo. A subembodiment of the invention of formula II is realized when Z is R. Another subembodiment of the invention of formula II is realized when Z is unsubstituted or substituted phenyl. Another subembodiment of the invention of formula II is realized when Z is unsubstituted or substituted pridyl. Another subembodiment of the invention of formula II is realized when Z is unsubstituted or substituted pyrimidinyl. Another subembodiment of the invention of formula II is realized when Z is unsubstituted or substituted pyrazinyl.

Another embodiment of the invention of formula II is realized when the (CHR) group of formula II is not present, thus it is represented as (CHR)₀.

Another embodiment of the invention of formula II is realized when the (CHR) group of formula II is present, thus it is represented as (CHR)₁.

Another embodiment of the invention of formula II is realized when the (CHR) group of formula II is present, and —NR(CHR)-Z is selected from the group consisting of N(CH₃)CH₂phenyl, N(CH₃)CH₂pridyl, N(CH₃)CH₂pyrimidinyl, N(CH₃)CH₂pyrazinyl, said phenyl, pyridyl, pyrimidinyl and pyrazinyl unsubstituted or substituted with 1 to 3 groups of R^{d}.

Another embodiment of the invention of formula II is realized when R² is hydrogen, R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}, and q is 0.

Another subembodiment of the invention of formula II is realized when R² is hydrogen, R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}, and q is 1. A subembodiment of this aspect of the invention is realized when q is 1 and R^{X} is selected from the group consisiting of CH₃, OCH₃, CF₃, SO₂CH₃, fluoro, and chloro. A further subembodiment of this aspect of the invention is realized when R^{X} is in the para position on the phenyl ring. Still another subembodiment of this aspect of the invention is realized when R^{X} is CF₃ in the para position of the phenyl ring. Another embodiment of the invention of formula II is realized when R² is selected from the group consisting of CH₃, CH₂CH₃, (CH₂)ₙCH(CH₃)₂, (CH₂)ₙOCH(CH₃)₂, (CH₂)ₙC₃₋₆cycloalkyl wherein the cycloalkyl is cyclopropyl, cyclobutyl or cyclopentyl, or (CHR)ₙC₆₋₁₀phenyl

Another embodiment of the invention of formula II is realized when R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}. Another subembodiment of this aspect of the invention is realized when one of R³ and R⁴ is optionally substituted phenyl. Another subembodiment of this aspect of the invention is realized when both of R³ and R⁴ are optionally substituted phenyl. Another subembodiment of this aspect of the invention is realized when R⁴ is optionally substituted phenyl. Still another subembodiment of this aspect of the invention is realized when R⁴ is optionally substituted phenyl and R³ is selected from the group consisting of isobutyl, isopentyl, (CH₂)ₙCF₃, (CH₂)ₙcyclopropyl, phenyl, said isobutyl, isopentyl, cyclopropyl and phenyl optionally substituted with 1 to 3 groups of R^{d}. Another subembodiment of this aspect of the invention is realized when R³ is optionally substituted phenyl. Still another subembodiment of this aspect of the invention is realized when R³ is optionally substituted phenyl and R⁴ is selected from the group consisting of isobutyl, isopentyl, (CH₂)ₙCF₃, (CH₂)ₙcyclopropyl, phenyl, said isobutyl, isopentyl, cyclopropyl and phenyl optionally substituted with 1 to 3 groups of R^{d}.

Another embodiment of the invention of formula I is realized when R^{d} is selected the group consisting of CF₃, fluoro, chloro, bromo, CN, C(O)NH₂, C(O)N(CH₃)₂, phenyl, pyridyl and furanyl, said phenyl, pyridyl and furanyl optionally substituted with 1 to 3 groups of halogen and CN.

All structural Formulas, embodiments and classes thereof described herein include the pharmaceutically acceptable salts of the compounds defined therein. Reference to the compounds of Formula I herein encompasses the compounds of formulas I, and II and all embodiments and classes thereof. Reference to the compounds of this invention as those of a specific formula or embodiment, e.g., formula I, and II or embodiments thereof, or any other generic structural formula or specific compound described or claimed herein, is intended to encompass the specific compound or compounds falling within the scope of the formula or embodiment, including salts thereof, particularly pharmaceutically acceptable salts, solvates (including hydrates) of such compounds and solvated salt forms thereof, where such forms are possible, unless specified otherwise

The present invention includes each of the Examples described herein, and pharmaceutically acceptable salts thereof. The invention also encompasses pharmaceutical compositions comprising an effective amount of a compound of the invention or a pharmaceutically acceptable salt thereof, and a pharmaceutically acceptable carrier.

As used herein, the term “alkyl” refers to a straight or branched chain, saturated aliphatic hydrocarbon radical having a number of carbon atoms in the specified range. Thus, for example, “—C₁₋₆ alkyl” (or “—C₁-C₆ alkyl”) means linear or branched chain alkyl groups, including all isomers, having the specified number of carbon atoms and includes each of the hexyl and pentyl isomers as well as each of n-, iso-, sec- and tert-butyl (butyl, s-butyl, i-butyl, t-butyl; Bu=butyl), n- and i-propyl (Pr=propyl), ethyl (Et) and methyl (Me). As another example, “—C₁₋₄ alkyl” refers to each of n-, iso-, sec- and t-butyl; n- and iso-propyl; ethyl and methyl. As another example, “—C₁₋₃ alkyl” refers to each of n-propyl, iso-propyl, ethyl and methyl. An alkyl group, when viewed in context within a chemical structure, may be univalent (e.g., when R^{2a} is unsubstituted —C₁₋₆ alkyl), bivalent (e.g., when R^{2a} is mono-substituted —C₁₋₆alkyl), or multi-valent (e.g., when R^{2a} is —C₁₋₆alkyl having two or more substituents).

The term “halogen” (or “halo”) refers to fluorine, chlorine, bromine and iodine (alternatively referred to as fluoro, chloro, bromo, and iodo). Fluoro or chloro are preferred.

“Cycloalkyl” is a cyclized alkyl ring having the indicated number of carbon atoms. Thus, for example, “—C₃₋₆ cycloalkyl” (or “—C₃-C₆ cycloalkyl”) refers to each of cyclopropyl, cyclobutyl, cyclopentyl, and cyclohexyl.

The term “haloalkyl” refers to an alkyl group as defined above in which one or more of the hydrogen atoms have been replaced (i.e., substituted) with a halogen (i.e., F, Cl, Br and/or I). Thus, for example, “—C₁₋₆ haloalkyl” (or “—C₁-C₆ haloalkyl”) refers to a —C₁ to C₆ linear or branched alkyl group as defined above with one or more halogen substituents; particularly 1-6 halogen substituents; and more particularly 1-3 halogen substituents. The term “fluoroalkyl” has an analogous meaning except that the halogen substituents are restricted to fluoro. Suitable fluoroalkyls include the series —(CH₂)₀₋₄CF₃ (i.e., trifluoromethyl, 2,2,2-trifluoroethyl, 3,3,3-trifluoro-n-propyl, etc.). A fluoroalkyl of particular interest is CF₃.

The term “C(O)” refers to carbonyl. The terms “S(O)₂” and “SO₂” each refer to sulfonyl. The term “S(O)” refers to sulfinyl.

As used herein ═O includes oxo (e.g., an annular —CH— substituted with oxo is —C(O) or carbonyl).

The term “aryl,” by itself or as part of another substituent, means an aromatic cyclic hydrocarbon radical. Preferred aryl groups have from six to ten carbons atoms. The term “aryl” includes multiple ring systems as well as single ring systems. Preferred aryl groups for use in the invention include phenyl and naphthyl.

The term “aryl” also includes fused cyclic hydrocarbon rings which are partially aromatic (i.e., one of the fused rings is aromatic and the other is non-aromatic). An exemplary aryl group which is partially aromatic is indanyl.

The term heterocyclyl, heterocycle or heterocyclic, as used herein, represents a stable 4- to 7-membered monocyclic, stable 8- to 11-membered bicyclic heterocyclic, or 8- to 13 tricyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic includes heteroaryl moieties and heterocycloalkyl moieties. Examples of such heterocyclic elements include, but are not limited to, azepinyl, benzodioxolyl, benzimidazolyl, benzisoxazolyl, benzofurazanyl, benzopyranyl, benzothiopyranyl, benzofuryl, benzothiazolyl, benzothienyl, benzotriazolyl, benzoxazolyl, chromanyl, cinnolinyl, dihydrobenzofuryl, dihydroisobenzofuranyl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyrazolopyridinyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl, and triazolyl.

The term “heteroaryl”, as used herein except where noted, represents a stable 5- to 7-membered monocyclic- or stable 9- to 11-membered fused bicyclic heterocyclic ring system which contains an aromatic ring. Any additional ring or rings fused to the aromatic ring may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure.

When a heterocyclyl group as defined herein is substituted, the substituent may be bonded to a ring carbon atom of the heterocyclic group, or on a ring heteroatom (i.e., a nitrogen, oxygen or sulfur), which has a valence which permits substitution. Preferably, the substituent is bonded to a ring carbon atom. Similarly, when a heterocyclic group is defined as a substituent herein, the point of attachment may be at a ring carbon atom of the heterocyclic group, or on a ring heteroatom (i.e., a nitrogen, oxygen or sulfur), which has a valence which permits attachment. Preferably, the attachment is at a ring carbon atom.

It is understood that the specific rings listed above are not a limitation on the rings which can be used in the present invention. These rings are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results.

Unless expressly stated to the contrary, all ranges cited herein are inclusive. For example, a heteroaromatic ring described as containing from “1 to 3 heteroatoms” means the ring can contain 1, 2 or 3 heteroatoms. It is also understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, or 4 heteroatoms. As another example, a moeity described as optionally substituted with “from 1 to 3 substituents” is intended to include as aspects thereof, such moeity substituted with 1 to 3 substituents, 2 or 3 substituents, 3 substituents, 1 or 2 substituents, 2 substituents, or 1 substituent.

When any variable (e.g., R³ or R^{3a}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

Unless expressly depicted or described otherwise, variables depicted in a structural formula with a “floating” bond attached to a ring, such as R^{a}, are permitted to be a substituent on any available carbon or nitrogen atom in the ring to which the variable is attached. When a moiety is noted as being “optionally substituted” in formula I or any embodiment thereof, it means that formula I or the embodiment thereof encompasses compounds that are substituted with the noted substituent (or substituents) on the moiety and compounds that do not contain the noted substituent (or substituents) on the moiety (i.e., wherein the moiety is unsubstituted).

Unless expressly stated to the contrary, substitution by a named substituent is permitted on any atom in a chain or ring provided such substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

To the extent substituents and substituent patterns provide for the existence of tautomers (e.g., keto-enol tautomers) in the compounds of the invention, all tautomeric forms of these compounds, whether present individually or in mixtures, are within the scope of the present invention. Compounds of the present invention having a hydroxy substituent on a carbon atom of a heteroaromatic ring are understood to include compounds in which only the hydroxy is present, compounds in which only the tautomeric keto form (i.e., an oxo substitutent) is present, and compounds in which the keto and enol forms are both present.

The compounds of Formula I may have one or more chiral (asymmetric) centers. The present invention encompasses all stereoisomeric forms of the compounds of Formula I. Centers of asymmetry that are present in the compounds of Formula I can all independently of one another have (R) or (S) configuration. When bonds to a chiral carbon are depicted as straight lines in the structural Formulas of the invention, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the Formula or by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. The present invention includes all such isomers, as well as salts, solvates (which includes hydrates) and solvated salts of such racemates, enantiomers, diastereomers and tautomers and mixtures thereof.

In the compounds of Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within Formula I can be prepared without undue experimentation by conventional techniques well known to those skilled in the art or by processes analogous to those described in the Schemes and Examples herein using appropriate isotopically-enriched reagents and/or intermediates.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof).

When the compounds of Formula I contain one or more acidic or basic groups the invention also includes the corresponding pharmaceutically acceptable salts. Thus, the compounds of Formula I which contain acidic groups can be used according to the invention as, for example but not limited to, alkali metal salts, alkaline earth metal salts or as ammonium salts. Examples of such salts include but are not limited to sodium salts, potassium salts, calcium salts, magnesium salts or salts with ammonia or organic amines such as, for example, ethylamine, ethanolamine, triethanolamine or amino acids. Compounds of Formula I which contain one or more basic groups, i.e. groups which can be protonated, can be used according to the invention in the form of their acid addition salts with inorganic or organic acids as, for example but not limited to, salts with hydrogen chloride, hydrogen bromide, phosphoric acid, sulfuric acid, nitric acid, benzenesulfonic acid, methanesulfonic acid, p-toluenesulfonic acid, naphthalenedisulfonic acids, oxalic acid, acetic acid, trifluoroacetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

Another embodiment of the present invention is a compound of Formula I, or a pharmaceutically acceptable salt thereof, as originally defined or as defined in any of the foregoing embodiments, aspects, classes, or subclasses, wherein the compound or its salt is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The compounds of the invention have two or more asymmetric centers and can occur as mixtures of stereoisomers. It is understood that a substantially pure compound can be either a substantially pure mixture of stereoisomers or a substantially pure individual diastereomer or enantiomer. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A compound or salt of 100% purity is one which is free of detectable impurities as determined by a standard method of analysis.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁₋₆alkyl esters and —C₁₋₆alkyl substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The invention also encompasses methods for the treatment or prophylaxis of infection by HIV or for the treatment, prophylaxis, or delay in the onset of AIDS in a subject in need thereof, which comprises administering to the subject an effective amount of a compound of the invention or a pharmaceutically acceptable salt thereof.

The invention also encompasses a compound of the invention, or a pharmaceutically acceptable salt thereof, for use in the preparation of a medicament for the inhibition of HIV protease, for the treatment or prophylaxis of infection by HIV, or for the treatment, prophylaxis, or delay in the onset of AIDS in a subject in need thereof.

The invention also encompasses a pharmaceutical composition comprising an effective amount of a compound of the invention, or a pharmaceutically acceptable salt thereof, and a pharmaceutically acceptable carrier and further comprising an effective amount of an anti-HIV agent selected from the group consisting of HIV antiviral agents, immunomodulators, and anti-infective agents. Within this embodiment, the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

Compounds of formula II and III each form a subset of the compounds included in formula I. Any description which follows that refers to a compound of Formula I also applies to a compound of formula II and all embodiments thereof.

Other embodiments of the present invention include the following:

(a) A pharmaceutical composition comprising an effective amount of a compound of formula I as defined above, or a pharmaceutically acceptable salt thereof, and a pharmaceutically acceptable carrier.

(b) A pharmaceutical composition which comprises the product prepared by combining (e.g., mixing) an effective amount of a compound of formula I as defined above, or a pharmaceutically acceptable salt thereof, and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

(e) The pharmaceutical composition of (d), wherein the antiviral is selected from the group consisting of HIV reverse transcriptase inhibitors and HIV integrase inhibitors.

(f) A combination which is (i) a compound of Formula I as defined above, or a pharmaceutically acceptable salt thereof, and (ii) an anti-HIV agent selected from the group consisting of HIV antiviral agents, immunomodulators, and anti-infective agents; wherein Compound I and the anti-HIV agent are each employed in an amount that renders the combination effective for inhibition of HIV protease, for treatment or prophylaxis of infection by HIV, or for treatment, prophylaxis of, or delay in the onset or progression of AIDS.

(g) The combination of (f), wherein the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

(h) The combination of (g), wherein the antiviral is selected from the group consisting of HIV reverse transcriptase inhibitors and HIV integrase inhibitors.

(i) A method for the inhibition of HIV protease in a subject in need thereof which comprises administering to the subject an effective amount of a compound of Formula I or a pharmaceutically acceptable salt thereof.

A method for the prophylaxis or treatment of infection by HIV (e.g., HIV-1) in a subject in need thereof which comprises administering to the subject an effective amount of a compound of Formula I or a pharmaceutically acceptable salt thereof.

(k) The method of (j), wherein the compound of Formula I is administered in combination with an effective amount of at least one other HIV antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

(1) The method of (k), wherein the at least one other HIV antiviral is selected from the group consisting of HIV reverse transcriptase inhibitors and HIV integrase inhibitors.

(m) A method for the prophylaxis, treatment or delay in the onset or progression of AIDS in a subject in need thereof which comprises administering to the subject an effective amount of a compound of Formula I or a pharmaceutically acceptable salt thereof

(n) The method of (m), wherein the compound is administered in combination with an effective amount of at least one other HIV antiviral, selected from the group consisting of HIV protease inhibitors, HIV reverse transcriptase inhibitors, HIV integrase inhibitors, HIV fusion inhibitors, HIV entry inhibitors, and HIV maturation inhibitors.

(o) The method of (n), wherein the at least one other HIV antiviral is selected from the group consisting of HIV reverse transcriptase inhibitors and HIV integrase inhibitors.

(p) A method for the inhibition of HIV protease in a subject in need thereof which comprises administering to the subject the pharmaceutical composition of (a), (b), (c) or (d) or the combination of (e) or (f).

(q) A method for the prophylaxis or treatment of infection by HIV (e.g., HIV-1) in a subject in need thereof which comprises administering to the subject the pharmaceutical composition of (a), (b), (c), (d) or (e).

(r) A method for the prophylaxis, treatment, or delay in the onset or progression of AIDS in a subject in need thereof which comprises administering to the subject the pharmaceutical composition of (a), (b), (c), (d) or (e).

The present invention also includes a compound of formula I, or a pharmaceutically acceptable salt thereof, (i) for use in, (ii) for use as a medicament for, or (iii) for use in the manufacture/preparation of a medicament for: (a) therapy (e.g., of the human body), (b) medicine, (c) inhibition of HIV protease, (d) treatment or prophylaxis of infection by HIV, or (e) treatment, prophylaxis of, or delay in the onset or progression of AIDS. In these uses, the compounds of the present invention can optionally be employed in combination with one or more other anti-HIV agents selected from HIV antiviral agents, anti-infective agents, and immunomodulators.

Additional embodiments of the invention include the pharmaceutical compositions, combinations and methods set forth in (a)-(r) above and the uses (i)(a)-(e) through (iii)(a)-(e) set forth in the preceding paragraph, wherein the compound of the present invention employed therein is a compound of one of the embodiments, aspects, classes or subclasses described above. In all of these embodiments, the compound can optionally be used in the form of a pharmaceutically acceptable salt.

Additional embodiments of the present invention include each of the pharmaceutical compositions, combinations, methods and uses set forth in the preceding paragraphs, wherein the compound of the present invention or its salt employed therein is substantially pure. With respect to a pharmaceutical composition comprising a compound of formula I or a pharmaceutically acceptable carrier and optionally one or more excipients, it is understood that the term “substantially pure” is in reference to a compound of formula I or its salt per se.

The methods of the present invention involve the use of compounds of the present invention in the inhibition of HIV protease (e.g., wild type HIV-1 and/or mutant strains thereof), the prophylaxis or treatment of infection by human immunodeficiency virus (HIV) and the prophylaxis, treatment or delay in the onset or progression of consequent pathological conditions such as AIDS. Prophylaxis of AIDS, treating AIDS, delaying the onset or progression of AIDS, or treating or prophylaxis of infection by HIV is defined as including, but not limited to, treatment of a wide range of states of HIV infection: AIDS, ARC (AIDS related complex), both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery.

In general, compounds that are HIV protease inhibitors can be identified as those compounds which, when tested in the “Cell-based HIV Infection Assay using a Reporter” assay described below, have an inflection point (IP) of 10 μM, particulalry 5 μM or less, preferably 1 μM or less, and more preferably 0.25 μM or less.

The term “administration” and variants thereof (e.g., “administering” a compound) in reference to a compound of formula I mean providing the compound to the individual in need of treatment or prophylaxis and includes both self-administration and administration to the patient by another person. When a compound is provided in combination with one or more other active agents (e.g., antiviral agents useful for treating or prophylaxis of HIV infection or AIDS), “administration” and its variants are each understood to include provision of the compound and other agents at the same time or at different times. When the agents of a combination are administered at the same time, they can be administered together in a single composition or they can be administered separately.

As used herein, the term “composition” is intended to encompass a product comprising the specified ingredients, as well as any product which results from combining the specified ingredients. By “pharmaceutically acceptable” is meant that the ingredients of the pharmaceutical composition must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” which is an amount effective for inhibiting HIV protease (wild type and/or mutant strains thereof), inhibiting HIV replication (either of the foregoing which may also be referred to herein as an “inhibition effective amount”), treating HIV infection, treating AIDS, delaying the onset of AIDS and/or slowing progression of AIDS. In another embodiment, the effective amount is a “prophylactically effective amount” which is an amount effective for prophylaxis of HIV infection or prophylaxis of AIDS. It is understood that an effective amount can simultaneously be both a therapeutically effective amount, e.g., for treatment HIV infection, and a prophylactically effective amount, e.g., for prevention or reduction of risk of developing AIDS. When the active compound (i.e., active ingredient) is administered as the salt, references to the amount of active ingredient are to the free form (i.e., the non-salt form) of the compound.

In the methods of the present invention (e.g., inhibiting HIV protease, treating or prophylaxis of HIV infection, inhibiting HIV replication, treating or prophylaxis of AIDS, delaying the onset of AIDS, or delaying or slowing progression of AIDS), the compounds of formula I, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered by one or more of the follwing routes: orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005.

The compounds of formula I can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In some cases, depending on the potency of the compound or the individual response, it may be necessary to deviate upwards or downwards from the given daily dose. Furthermore, the compound may be formulated for immediate or modified release such as extended or controlled release.

As noted above, the present invention is also directed to use of a compound of formula I with one or more additional anti-HIV agents. An “anti-HIV agent” is any agent which is directly or indirectly effective in the inhibition of HIV reverse transcriptase, protease, or another enzyme required for HIV replication or infection, the inhibition of HIV replication, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, imunomodulators, antiinfectives, or vaccines useful for treating HIV infection or AIDS, such as those disclosed in Table 1 of WO 01/38332 or in the Table in WO 02/30930. Suitable HIV antivirals for use in combination with the compounds of the present invention include, for example, those listed in Table A as follows:

TABLE A
Antiviral Agents for Treating HIV infection or AIDS

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Revataz ® | PI |
| AZT, zidovudine, azidothymidine, Retrovir ® | nRTI |
| capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| dolutegravir, Tivicay ® | InI |
| doravirine, MK-1439 | nnRTI |
| efavirenz, EFV, Sustiva ®, Stocrin ® | nnRTI |
| efavirenz + emtricitabine + tenofovir DF, Atripla ® | nnRTI + nRTI |
| EFdA (4′-ethynyl-2-fluoro-2′-deoxyadenosine) | nRTI |
| Elvitegravir | InI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| Rilpivirine | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tipranavir, Aptivus ® | PI |
| vicriviroc | EI |

EI = entry inhibitor; FI = fusion inhibitor; InI = integrase inhibitor; PI = protease inhibitor; nRTI = nucleoside reverse transcriptase inhibitor; nnRTI = non-nucleoside reverse transcriptase inhibitor. Some of the drugs listed in the table are used in a salt form; e.g., abacavir sulfate, delavirdine mesylate, indinavir sulfate, atazanavir sulfate, nelfinavir mesylate, saquinavir mesylate.

It is understood that the scope of combinations of the compounds of this invention with anti-HIV agents is not limited to the HIV antivirals listed in Table A and/or listed in the above-referenced Tables in WO 01/38332 and WO 02/30930, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), or the 59^{th} edition (2005) and the current Physicians' Desk Reference (68th ed.). (2014), Montvale, N.J.: PDR Network. The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV protease, e.g., by competitive inhibition. Thus the compounds of this invention are commercial products to be used for these purposes.

Abbreviations and acronymns employed herein include the following: Bn=benzyl; BOC (or Boc)=t-butyloxycarbonyl; Boc₂O =di-t-butyl carbonate; BOP=benzotriazol-1-yloxytris-(dimethylamino)phosphonium; BSA=bovine serum albumin; CBS=Corey, Bakshi, Shibata chiral oxazaborolidine mediated ketone reduction; Cbz=benzyloxycarbonyl; DBU=1,8-diazabicyclo[5.4.0]undec-7-one; DCAD=di-(4-chlorobenzyl) azodicarboxylate; DCE=1,2-dichloroethane; DCM=dichloromethane; DEAD=diethyl azodicarboxylate; DIAD=diisopropylazodicarboxylate; Dibal-H=diisobutylaluminum hydride; DMAP=4-dimethylaminopyridine; DMF=dimethylformamide; DMSO=dimethyl sulfoxide; e.g.=for example (but not limited to); EDC=1-ethyl-3-(3-dimethylaminopropyl) carbodiimide; Et=ethyl; EtOAc=ethyl acetate; EtOH=ethanol; G-2G=Grubbs catalyst, 2^{nd} generation; HOAt=1-hydroxy-7-azabenzotriazole; HPLC=high performance liquid chromatography; HSU=hydroxysuccinimide; i-PrOH=isopropanol; LAH=lithium aluminum hydride; LCMS=liquid chromatography-mass spectroscopy; Me=methyl; MeOH=methanol; MOC=methoxycarbonyl; Ms=mesyl or methanesulfonyl; NMR=nuclear magnetic resonance; Ph=phenyl; RCM=ring closing metathesis; Piv=pivaloyl; PPTS=pyridinium p-toluene sulfonate; PyBrOP=bromo-tris-pyrrolidinophosphonium hexafluorophosphate;); rt, r.t. or RT=room temperature; SCX=strong cation exchange resin; STP=standard temperature and pressure (i.e., 25° C. & 1 atmosphere); TBS=tert-butyldimethylsilyl; TBDPS=tert-butyl(diphenyl) silyl; TBDPSC1=tert-butyl(dimethyl)silyl chloride; TEA=triethylamine; TFA=trifluoroacetic acid; THF=tetrahydrofuran; TLC=thin layer chromatography; TMAF=tetramethyl ammonium fluoride; TMSCHN₂=trimethylsilyl diazomethane; TPAP=tetrapropylammonium perruthenate; TPP=triphenylphosphine.

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. In the examples that follow, when a nitrogen atom is depicted without the necessary hydrogen atoms to complete the valence, it is assumed those hydrogen atoms are present unless specifically stated to the contrary.

This invention relates to the preparation and use of compounds represented by Formula I:

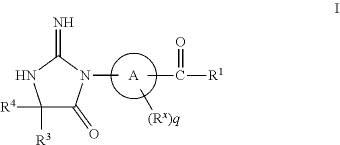

The compounds of formula I can be prepared using the general synthetic reaction schemes shown in Methods A to C.

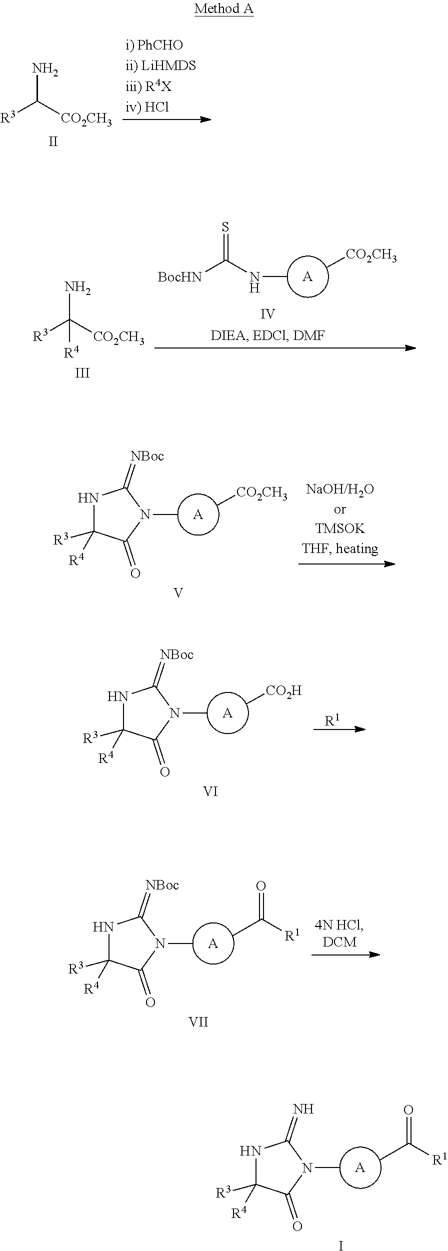

Method A provides a route to compounds V and then to formula I compounds by first elaborating the monosubstituted amino acid ester II to the requisite disubstitiuted amino ester III. For example II is reacted with benzaldehyde under dehydrating conditions to provide an intermediate imine which can be treated with a strong base such as LiHMDS or LDA and reacted with an alkylating agent such as a alkyl halide or triflate (R⁴X) followed by subsequent hydrolysis of the imine to give di-substituted amino acid esters III. Condensation of III with with thioureas IV provide compounds V in a similar manner as described in the literature (see McKittrick et al, Bioorganic & Medicinal Chemistry Letters (2015), 25(7), 1592-1596 and references therein) using a coupling agent such as a carbodiimide under standard peptide coupling conditions. The resultant compounds V are saponified to their corresponding carboxylic acids VI. Acids VI are condensed under standard peptide coupling conditions with amines represented by structure R¹ to provide amides VII. The resultant amides are then treated with an acid such as TFA or HCl to remove the BOC protecting group to provide compounds of formula I.

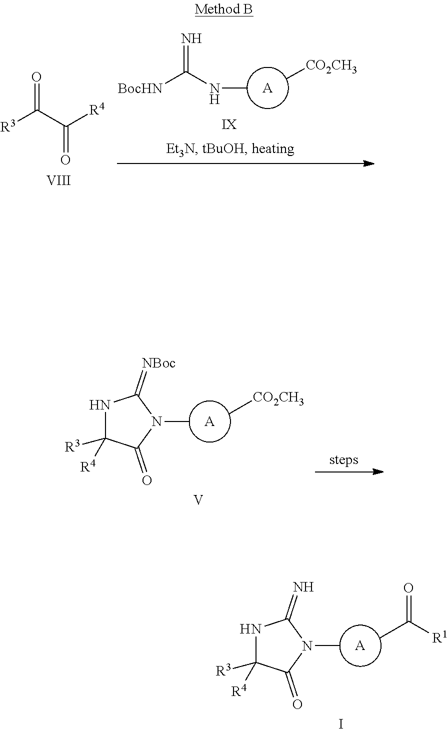

Alternatively, esters V can be prepared by condensing alpha diketones VIII with substituted guanidines IX according to literature procedures. The compounds V are then converted to compounds of formula I according to method A.

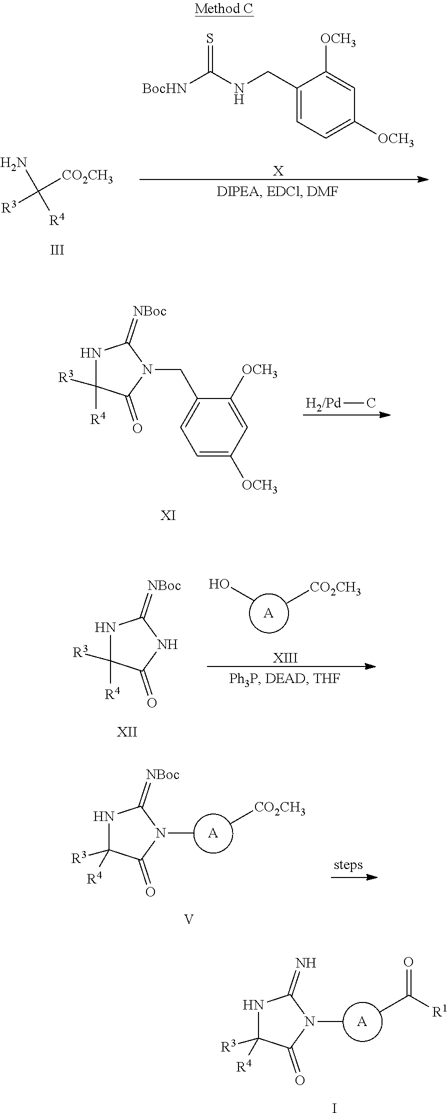

Method C provides another route to compounds V and then to formula I compounds by first condensing the disubstituted amino acid ester III with orthogonally protected thioureas X. For example, one embodiment of an orthogonally protected thiourea has a BOC protecting group on one nitrogen and a dimethoxybenzyl protecting group on the other nitrogen. The resultant compounds XI are then treated with palladium under an atmosphere of hydrogen to provide intermediates XII. Reaction of compounds XII with suitable alcohols XIII under Mitsunobu conditions provides compounds V and then compounds of formula I according to the appropriate steps from method A.

Detailed procedures for specific illustrative examples are shown below.

### Method A, Scheme 1:

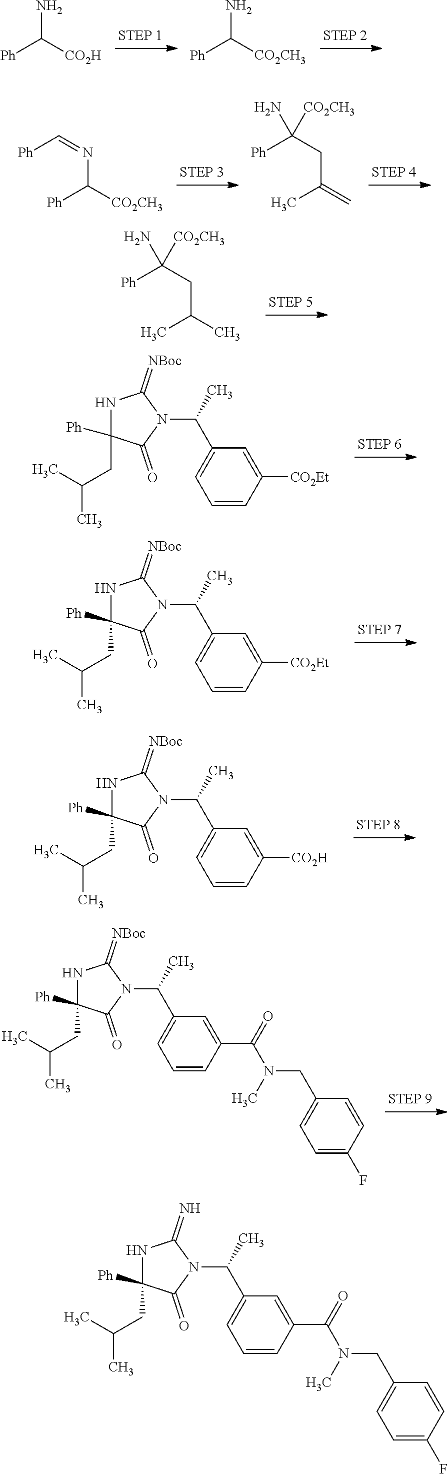

### Scheme 1, Step 1:

Methyl 2-amino-2-phenylacetate

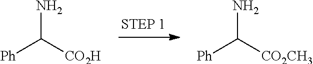 [SMILES: NC(C(=O)O)c1ccccc1 ; COC(=O)C(N)c1ccccc1]

To a cooled solution of phenylglycine (40.3 g, 266.3 mmol) in MeOH (250 ml), was added thionyl chloride (29.0 mL, 399.5 mmol) dropwise and stirred for 12 h until a colorless solution was obtained. The solvents were evaporated to give a pale yellow solid, more methanol was added to dissolve the solid and the solution was evaporated to dryness. The solid was placed on a high vacuum pump for 24 h. (53.0 g). ¹H NMR (300 MHz, CD₃OD) δ7.51 (d, J=1.2 Hz, 5H), 5.22 (s, 1H), 3.8 (s, 3H).

### Scheme 1, Step2:

Methyl 2-(benzylideneamino)-2-phenylacetate

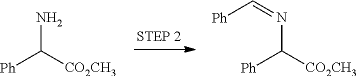 [SMILES: COC(=O)C(N)c1ccccc1 ; COC(=O)C(/N=C\c1ccccc1)c1ccccc1]

To a suspension of methyl 2-amino-2-phenylacetate (53.0 g, 263 mmol) in dichloromethane (250 mL) was added Et₃N (44.7 mL, 315 mmol) dropwise and stirred for 1 h. Benzaldehyde (27 mL, 263 mmol) was added and the reaction was stirred for 12 h. Water (50 mL) was added to the reaction mixture and transferred to a separatory funnel. The organic layer was washed with brine and dried over Na₂SO₄, filtered, and concentrated to give an oil which eventually solidified on the high vacuum pump to afford methyl 2-(benzylideneamino)-2-phenylacetate (65.9 g). ¹H NMR (300 MHz, CDCl₃) δ10.02 (s, 1H), 7.84-7.79 (m, 2H) 7.53 (d, J=1.5 Hz) 7.5-7.28 (m, 8H), 5.20 (s, 1H), 3.74 (s, 3H).

### Scheme 1, Step 3:

Methyl 2-amino-4-methyl-2-phenylpent-4-enoate

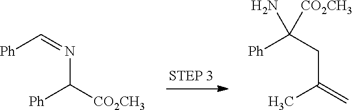 [SMILES: COC(=O)C(/N=C\c1ccccc1)c1ccccc1 ; C=C(C)CC(N)(C(=O)OC)c1ccccc1]

To an oven dried round bottom flask was added methyl 2-(benzylideneamino)-2-phenylacetate (5.0 g, 19.7 mmol), THF (75 mL), and 3-bromo-2-methylpropene (3.2 g, 23.9 mmol) and the mixture was cooled to −78° C. LiHMDS (45 mL, 1M THF solution, 45 mmol) was added dropwise and stirred for 12 h as the reaction mixture warmed to room temperature. The mixture was cooled in an ice bath as 2N HCl (2×30 mL) was added then transferred to a separatory funnel. The aqueous layer was separated and basified to pH 8-10 with 2N NaOH. The mixture was transferred back to the separatory funnel and extracted with EtOAc (3×30 mL). Then the organic portion was dried over Na₂SO₄, filtered, and concentrated to give an oil. The oil was purified by silica gel chromatography (0-30% EtOAc/Hexanes) to give methyl 2-amino-4-methyl-2-phenylpent-4-enoate (1.45 g). ¹H NMR (300 MHz, CDCl₃) δ7.60-7.51 (m, 2H), 7.37-7.27 (m, 3H), 4.93 (t, J=1.6 Hz,1H), 4.80 (d, J=0.7 Hz, 1H), 3.71 (s, 3H), 3.11 (d, J=13.7 Hz, 1H), 2.69 (d, J=13.3 Hz, 1H), 2.04 (bs, 2H), 1.28 (s, 3H).

### Scheme 2, Step 4:

Methyl 2-amino-4-methyl-2-phenylpentanoate

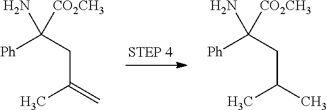 [SMILES: C=C(C)CC(N)(C(=O)OC)c1ccccc1 ; COC(=O)C(N)(CC(C)C)c1ccccc1]

To a solution methyl 2-amino-4-methyl-2-phenylpent-4-enoate (2.8 g, 12.77 mmol) in EtOH (100 mL) was added 10 wt. % Pd/C (0.7 g). The mixture was evacuated, then purged under nitrogen, then switched to hydrogen. After 17 h, the reaction mixture was purged with nitrogen, filtered, and concentrated to give compound methyl 2-amino-4-methyl-2-phenylpentanoate (2.8 g). ¹H NMR (300 MHz, CDCl₃) δ7.55-7.50 (m, 2H), 7.36-7.22 (m, 3H), 3.69 (s, 3H), 2.15-2.00 (m, 1H), 1.98-1.94 (m, 1H), 1.84-1.71 (m, 1H), 0.90 (d, J=6.7 Hz, 3H) 0.84 (d, J=6.6 Hz, 3H).

### Scheme 1, Step 5:

Ethyl 3-(1R)-1-(2-((tert-butoxycarbonyl)imino)-4-isobutyl-5-oxo-4-phenylimidazolidin-1-yl)ethyl)Benzoate

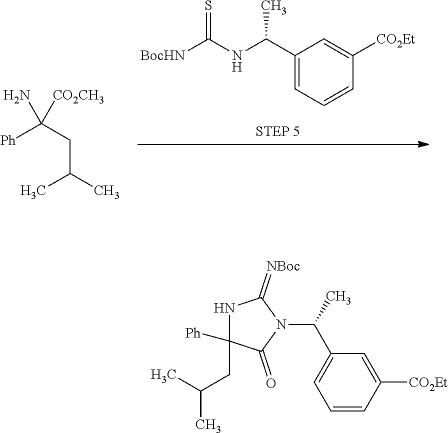

To a solution of methyl 2-amino-4-methyl-2-phenylpent-4-enoate (111 mg, 0.50 mmol) in DMF (3.1 mL) was added the (R)-ethyl-3-(1-(3-tert-butoxycarbonyl)thioureido)ethyl)benzoate (263 mg, 0.75 mmol), EDCI (129 mg, 0.68 mmol), and DIPEA (0.26 mL, 1.49 mmol). The mixture stirred at room temperature for 18 h then heated to 50° C. for an additional 4 h. The mixture was cooled to room temperature and then diluted with EtOAc (20 mL) then water (10 mL) and transferred to a separatory funnel. The EtOAc was washed with brine (3×10 mL) then dried over Na₂SO₄, filtered, and concentrated. The compound was further purified by silica gel chromatography, eluting with 0-50%:EtOAc in hexanes to give methyl 3-((2-((tert-butoxycarbonyl)imino)-4-(2-methylallyl)-5-oxo-4-phenylimidazolidin-1-yl)methyl)benzoate (118 mg) as a mixture of diasteromers. Mass Spec. (ESI+) m/z=508.3 (M+H⁺)

### Scheme 1, Step 6:

Ethyl 3-((R)-1-((R)-2-((tert-butoxycarbonyl)imino)-4-isobutyl-5-oxo-4-phenylimidazolidin-1-yl)ethyl)benzoate

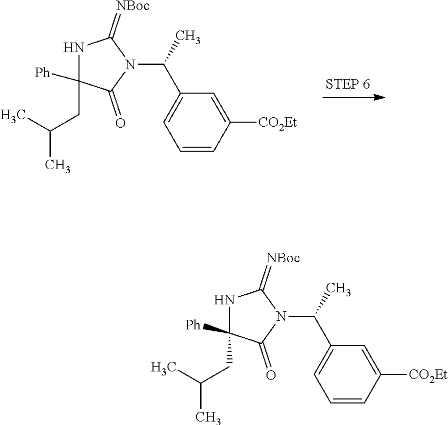

The mixture of diasteriomers of methyl 3-((2-((tert-butoxycarbonyl)imino)-4-(2-methylallyl)-5-oxo-4-phenylimidazolidin-1-yl)methyl)benzoate (118 mg) was resolved by chiral hplc on a Lux cellulose-2 column (10 μ, 30×250 mm) eluting with 3.2% isopropanol in hexanes with to afford ethyl 3-((R)-1-((S)-2-((tert-butoxycarbonyl)imino)-4-isobutyl-5-oxo-4-phenylimidazolidin-1-yl)ethyl)benzoate as the first eluting peak and ethyl 3-((R)-1-((R)-2-((tert-butoxycarbonyl)-imino)-4-isobutyl-5-oxo-4-phenylimidazolidin-1-yl)ethyl)benzoate as the second eluting peak.

### Scheme 1, Step 7:

3-((R)-1-((R)-2-((tert-butoxycarbonyl)imino)-4-isobutyl-5-oxo-4-phenylimidazolidin-1-yl)ethyl)Benzoic Acid

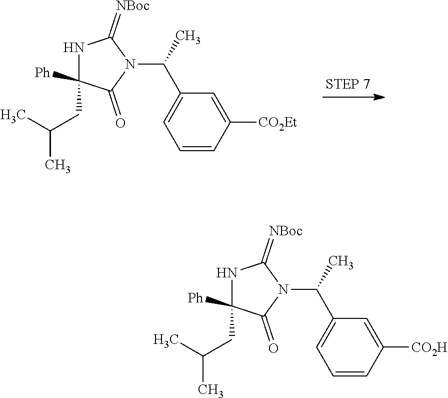

A suspension of ethyl 3-((R)-1-((R)-2-((tert-butoxycarbonyl)imino)-4-isobutyl-5-oxo-4-phenylimidazolidin-1-yl)ethyl)benzoate (54.8 mg, 0.11 mmol) and potassium trimethylsilanolate (62.5 mg, 0.44 mmol) in THF (2.5 mL) was heated to 50° C. for 2 h. The mixture stirred overnight at room temperature concentrated to dryness. EtOAc (50 mL) was added to the residue and 2N NaOH (30 mL). This mixture was transferred to a separatory funnel and the aqueous layer was separated, cooled in an ice bath, and the pH adjusted to to 5-6 with 2N HCl. The aqueous layer was added back to the EtOAc layer and, separated, and the aqueous layer was extracted with EtOAc (2×20 mL). The combined organic layers were dried over Na₂SO₄, filtered and concentrated to afford 3-((R)-1-((R)-2-((tert-butoxycarbonyl)imino)-4-isobutyl-5-oxo-4-phenylimidazolidin-1-yl)ethyl)benzoic acid (51.6 mg). Mass Spec. (ESI+) m/z=480.3 [M+H]⁺

### Scheme 1, Step 8:

tert-Butyl ((R)-1-((R)-1-(3-((4-fluorobenzyl)(methyl)carbamoyl)phenyl)ethyl)-4-5-isobutyl-5-oxo-4-phenylimidazolidin-2-ylidene)carbamate

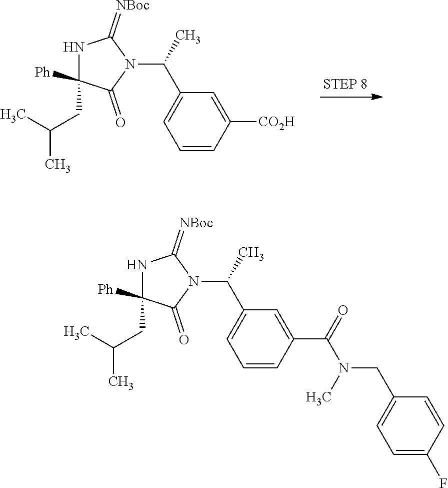

To a solution of 3-((R)-1-((R)-2-((tert-butoxycarbonyl)imino)-4-isobutyl-5-oxo-4-phenylimidazolidin-1-yl)ethyl)benzoic acid (9.9 mg, 0.02 mmol) in acetonitrile (750 μL) and THF (250 μL) was added polymer-supported EDC (loading 1.39 mmol/g; 100 mg, 0.14 mmol), HOBt (4.3 mg, 0.03 mmol), then 4-fluoro-N-methylbenzylamine (Aldrich) (4.2 μL, 0.03 mmol) and then agitiated for 20 h at room temperature. After this time, polymer-supported trisamine (loading 4.46 mmol/g; 27 mg, 0.12 mmol) and polymer-supported isocyanate (loading 1.46 mmol/g; 55 mg, 0.08 mmol) and acetonitrile (500 μL) was added to the mixture and agitated for 20 h at room temperature. Filter off the solids and wash with acetonitrile (2 mL) and THF (2 mL). Concentrate the combined organics to produce tert-butyl ((R)-1-((R)-1-(3-((4-fluorobenzyl)(methyl)carbamoyl)phenyl)ethyl)-4-isobutyl-5-oxo-4-phenylimidazolidin-2- ylidene)carbamate (10.5 mg) Mass Spec. (ESI+) m/z=601 [M+H]⁺.

### Scheme 1, Step 9:

N-(4-Fluorobenzyl)-3-((R)-1-((R)-2-imino-4-isobutyl-5-oxo-4-phenylimidazolidin-1-yl)ethyl)-N-methylbenzamide

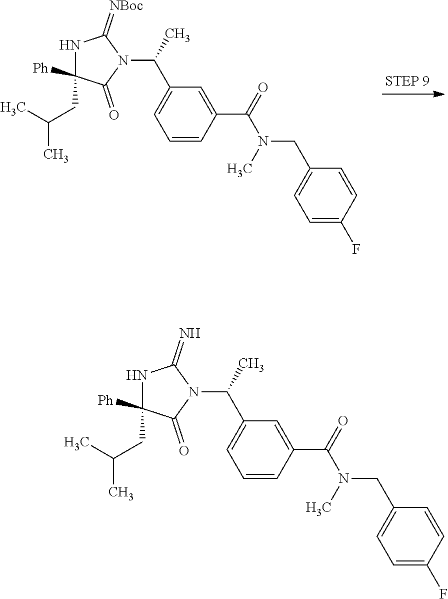

A solution of tert-butyl ((R)-1-((R)-1-(3-((4-fluorobenzyl)(methyl)carbamoyl)phenyl)ethyl)-4-isobutyl-5-oxo-4-phenylimidazolidin-2-ylidene)carbamate (10.5 mg, 0.017 mmol) in DCM (1 mL) was treated with TFA (200 μL) and agitated at room temperature for 2 h. The organic portion was concentrated in vacuo then reconstituted in DCM (0.5 mL) and methanol (0.5 mL). To this solution was added polymer-supported TsOH (loading 4.13 mmol/g; 150 mg, 0.62 mmol) and the resultant mixture was agitiated for 90 min at room temperature. The solvent was filtered off, and the polymer-supported TsOH was washed with DCM (2×1 mL) then MeOH (3×1 mL). The polymer-supported TsOH was then treated with 2 M ammonia in methanol solution (1.5 mL) for 45 min at room temperature. After this time, the solution was filtered, the resin was washed with methanol (5×1 mL), and the combined organic portions were concnetrated in vacuo to afford N-(4-fluorobenzyl)-3-((R)-1-((R)-2-imino-4-isobutyl-5-oxo-4-phenylimidazolidin-1-yl)ethyl)-N-methylbenzamide (8.7 mg) Mass Spec. (ESI+) m/z=501.3 [M+H]⁺.

### Method B, Scheme 2

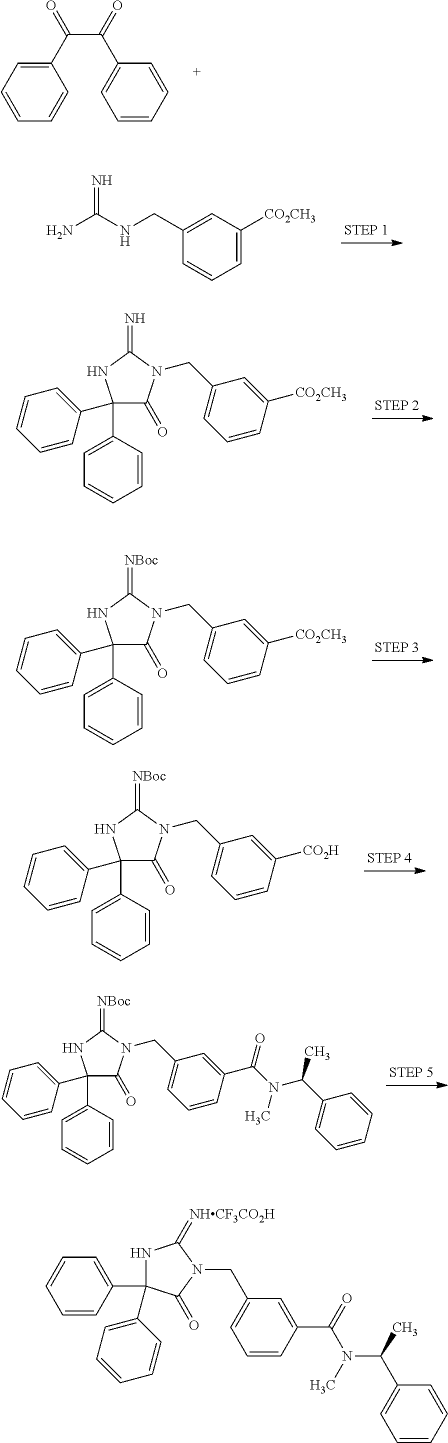

### Scheme 2, Step 1:

Methyl 3-((2-imino-5-oxo-4,4-diphenylimidazolidin-1-yl)methyl)benzoate

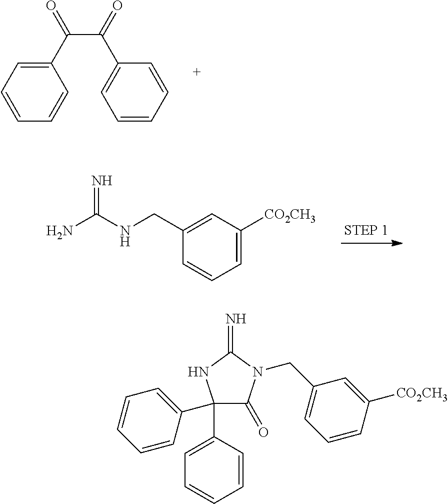

A mixture of benzil (1.56 g, 7.42 mmol), methyl 3-(guanidinomethyl)benzoate (2.86 g, 11.2 mmol) and Et₃N (4.1 mL, 29.7 mmol) in anhydrous ethanol (55 mL) was heated to refluxing temperature for 20 h under nitrogen. After this period the reaction mixture was cooled to room temperature and the precipitate was filtered off. The filtrate was concentrated under reduced pressure and the crude mixture was purified by silica gel chromatography eluting with 0 to 10% MeO H in DCM to furnish methyl 3-((4-(3-bromophenyl)-4-(4-fluorophenyl)-2-imino-5-oxoimidazolidin-1-yl)methyl)benzoate (1.02 g). Mass Spec. (APCI+) m/z=400 [M+H]⁺.

### Scheme 2, Step 2:

Methyl 3-((2-((tert-butoxycarbonyl)imino)-5-oxo-4,4-diphenylimidazolidin-1-yl)methyl)Benzoate

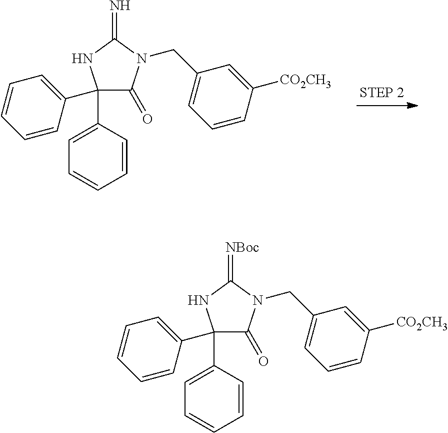

To a solution of methyl 3-((4-(3-bromophenyl)-4-(4-fluorophenyl)-2-imino-5-oxoimidazolidin-1-yl)methyl)benzoate (1.02 g, 2.55 mmol) in a mixture of 1-4-dioxane (12 mL), methanol (4 mL), and water (4 mL) at room temperature, was added (Boc)₂O (670 mg, 3.07 mmol) and sodium bicarbonate (322 mg, 3.83 mmol) under nitrogen. The reaction mixture was allowed to stir at room temperature and stirred for 16 h. After this period solvent was evaporated and residue was re-dissolved in EtOAc (20 mL). The organic layer was washed with water (2×20 mL) and brine (10 mL), dried over Na₂SO₄, filtered and concentrated to dryness. The crude mixture was purified by silica gel chromatography eluting with 0 to 100% EtOAc in hexanes to afford methyl 3-((2-((tert-butoxycarbonyl)imino)-5-oxo-4,4-diphenylimidazolidin-1-yl)methyl)benzoate (1.1 g) MS (ESI+) m/z 500 [M+H]⁺.

### Scheme 2, Step 3:

3-((2-((tert-butoxycarbonyl)imino)-5-oxo-4,4-diphenylimidazolidin-1-yl)methyl)Benzoic Acid

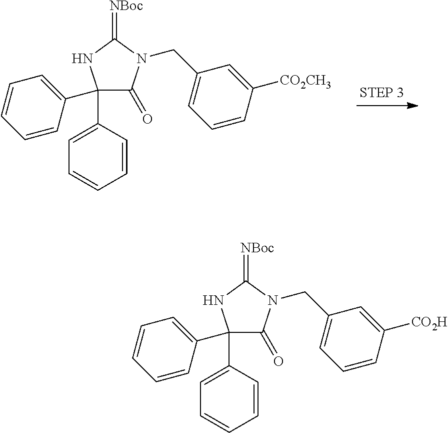

To a solution of methyl 3-((2-((tert-butoxycarbonyl)imino)-5-oxo-4,4-diphenylimidazolidin-1-yl)methyl)benzoate (1.1 g, 2.20 mmol) in THF (16 mL), was added potassium trimethylsilanolate 0.5 g, 3.96 mmol) and the reaction mixture was heated at 40° C. for 20 h under nitrogen. After this period the solvent was evaporated, then EtOAc (20 mL) and water (20 mL) added. The organic portion was washed with water, then brine. The combined organic portion was dried over sodium sulfate, filtered, and concentrated in vacuo to afford 3-((2-((tert-butoxycarbonyl)imino)-5-oxo-4,4-diphenylimidazolidin-1-yl)methyl)benzoic acid (1.0 g MS (ESI+) m/z 486 [M+H]+.

### Scheme 2, Step 4:

tert-Butyl (R)-(1-(3-(methyl(1-phenylethyl)carbamoyl)benzyl)-5-oxo-4,4-diphenylimidazolidin-2-ylidene)Carbamate

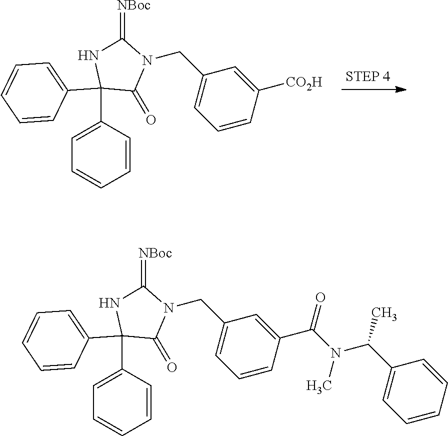

To a mixture 3-((2-((tert-butoxycarbonyl)imino)-5-oxo-4,4-diphenylimidazolidin-1-yl)methyl)benzoic acid (50 mg, 0.10 mmol), (R)-N-methyl-1-phenylethan-1-amine (18 mg, 0.12 mmol), and HATU (51 mg, 0.13 mmol) in anhydrous DMF (2 mL) was added DIPEA (27 μL, 0.15 mmol) and the reaction mixture stirred for 2 h at room temperature. After this period DMF evaporated, added EtOAc, then extracted with 1N HCl aq, water, then brine. The combined organic layer was dried over Na₂SO₄, filtered, and concentrated to dryness. The crude product was used in the next step without further purification MS (ESI+) m/z 602 [M+H]⁺.

### Scheme 2, Step 5:

(R)-3-((2-Imino-5-oxo-4,4-diphenylimidazolidin-1-yl)methyl)-N-methyl-N-(1-phenylethyl)benzamide 2,2,2-trifluoroacetate

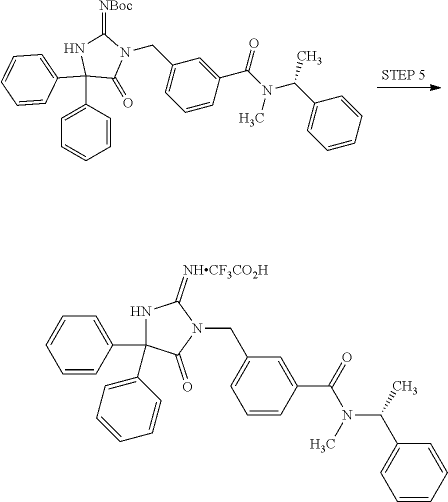

A solution of tert-butyl (R)-(1-(3-(methyl(1-phenylethyl)carbamoyl)benzyl)-5-oxo-4,4-diphenylimidazolidin-2-ylidene)carbamate in DCM (1 mL) was treated with 200 mL of TFA and stirred for 2 h. The solution was concentrated in vacuo and the residue was purified by reverse phase chromotagraphy (C18, eluting with 10 to 100% acetonitrile in water, 1% TFA modifier) to afford (R)-3-((2-imino-5-oxo-4,4-diphenylimidazolidin-1-yl)methyl)-N-methyl-N-(1-phenylethyl)benzamide 2,2,2-trifluoroacetate (22 mg, over two steps). Mass Spec. (ESI+) m/z=503.2 [M+H]⁺.

### Method C, Scheme 3

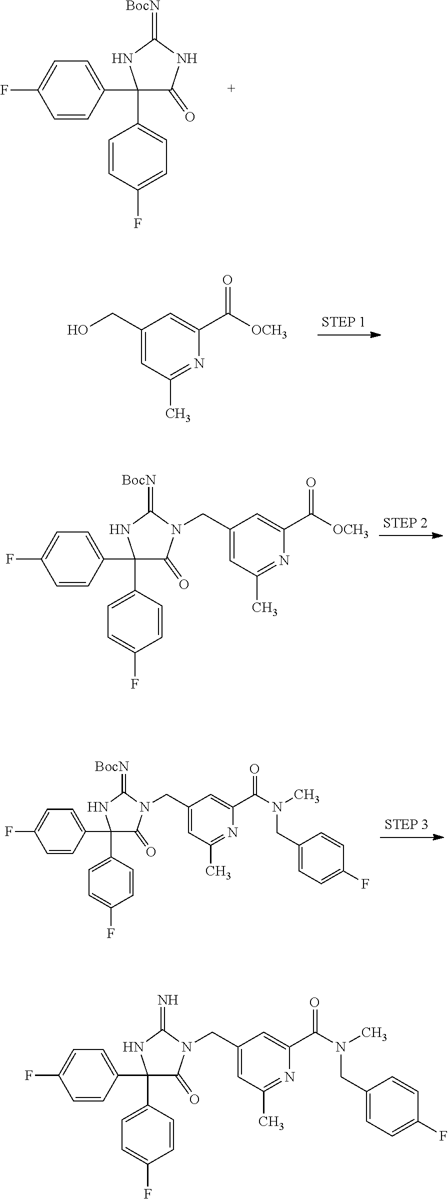

### Scheme 3, Step 1:

Methyl 4-((2-((tert-butoxycarbonyl)imino)-4,4-bis(4-fluorophenyl)-5-oxoimidazolidin-1-yl)methyl)-6-methylpicolinate

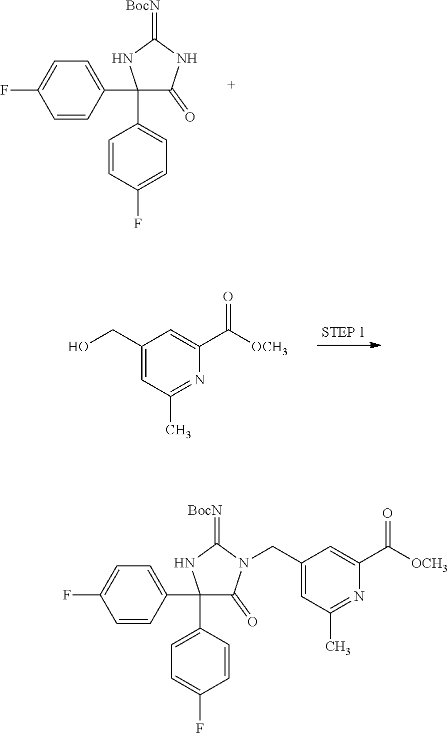

To a solution of isopropyl 5-(1-hydroxypentyl)-2-(trifluoromethyl)benzoate (95 mg, 0.3 mmol) and tert-butyl (R)-(4-(4-fluorophenyl)-4-isopentyl-5-oxoimidazolidin-2-ylidene)carbamate (91 mg, 0.25 mmol) in THF (1 mL) were added n-Bu₃P (0.1 mL, 0.40 mmol) and diethyl azodicarboxylate (0.078 mL, 0.50 mmol) at room temperature. The reaction mixture was stirred at room temperature under nitrogen for 12 h. The reaction mixture was concentrated down and purified by column chromatography using EtOAc and hexanes as eluents to yield isopropyl 5-(1-((R)-2-((tert-butoxycarbonyl)imino)-4-(4-fluorophenyl)-4-isopentyl-5-oxoimidazolidin-1- yl)pentyl)-2-(trifluoromethyl)benzoate (67 mg) as an oil. Mass Spec. (ESI+) m/z=664.4 [M+H]⁺.

### Scheme 3, Step 2:

tert-Butyl (1-((2-((04-fluorobenzyl)(methyl)carbamoyl)-6-methylpyridin-4-yl)methyl)-4,4-bis(4-fluorophenyl)-5-oxoimidazolidin-2-ylidene)carbamate

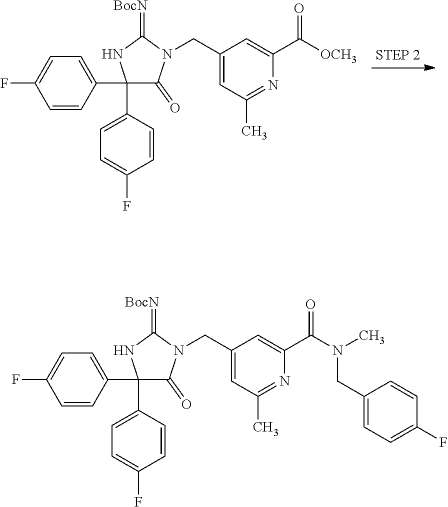

To a solution of bis(trimethylaluminum)-1,4-diazabicyclo[2.2.2]-octane (Aldrich) (60 mg, 0.23 mmol) in anhydrous THF (10 mL) was added 4-fluoro-N-methylbenzylamine (Aldrich) (29 μL, 0.22 mmol) and this solution was heated to 40° C. for 1 h. To this mixture was added methyl 4-((2-((tert-butoxycarbonyl)imino)-4,4-bis(4-fluorophenyl)-5-oxoimidazolidin-1-yl)methyl)-6-methylpicolinate (81 mg, 0.15 mmol) and heated to 55° C. for 16 h. The reaction mixture was cooled to room temperature then quenched with 2 M HCl aq dropwise. The mixture was extracted with diethyl ether, the organic portion was dried over sodium sulfate, filtered, and concentrated in vacuo. The mixture was further purified by silica gel chromatography eluting with 0 to 100% EtOAc in hexanes) to afford tert-butyl (1-((2-((4-fluorobenzyl)(methyl) carbamoyl)-6-methylpyridin-4-yl)methyl)-4,4-bis(4-fluorophenyl)-5-oxoimidazolidin-2-ylidene)carbamate which was used in the next step (16.7 mg) Mass Spec. (ESI+) m/z =658.2 [M+H]⁺.

### Scheme 3, Step 3:

4-((4,4-Bis(4-fluorophenyl)-2-imino-5-oxoimidazolidin-1-yl)methyl)-N-(4-fluorobenzyl)-N,6-dimethylpicolinamide Hydrochloride

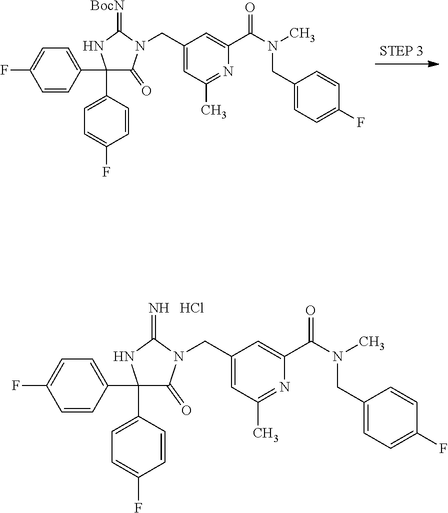

To a solution of tert-butyl (1-((2-((4-fluorobenzyl)(methyl)carbamoyl)-6-methylpyridin-4-yl)methyl)-4,4-bis(4-fluorophenyl)-5-oxoimidazolidin-2-ylidene)carbamate (16.7 mg, 0.025 mmol) in DCM (2 mL) was added TFA (400 μL). The reaction mixture was stirred at room temperature for 2 h. Then, the reaction mixture was concentrated and purified by reverse phase hplc chromatography (C18, eluting with 10 to 100% acetonitrile in water, 1% TFA modifier). The purified product was concentrated and converted to its hydrochloride salt by treatment with 1N HCl in acetonitrile (1 mL) to and this mixture was freeze dried and lyopholized to afford 4-((4,4-bis(4-fluorophenyl)-2-imino-5-oxoimidazolidin-1-yl)methyl)-N-(4- fluorobenzyl)-N,6-dimethylpicolinamide. (14 mg). Mass Spec. (ESI+) m/z=558.2. [M+H]⁺.

### Preparation of Intermediates

### Preparation of Compounds IV.

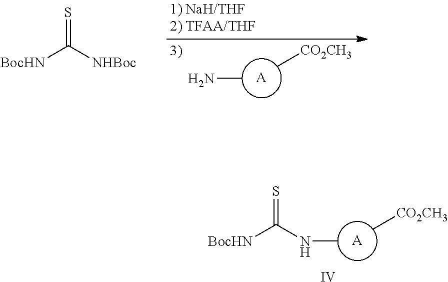

Compounds IV were generally prepared in a similar method described in the literature (reference: Synthesis 2010, 6, 991). A solution of N,N-bisboc-thiourea was treated with sodium hydride followed by treatment with TFAA. After which a solution of requisite amine was added to the reaction mixture to afford compound IV.

An example of the preparation of compound IV is shown below in Scheme 4.

### Scheme 4:

Methyl 3-((3-(tert-butoxycarbonyl)thioureido)methyl)-5-fluorobenzoate

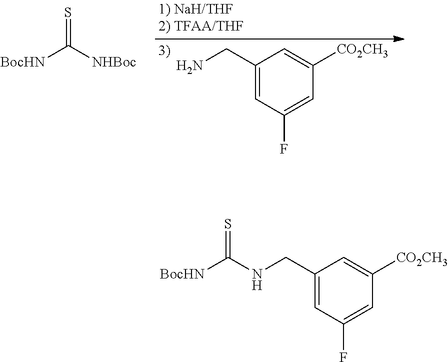 [SMILES: CC(C)(C)OC(=O)NC(=S)NC(=O)OC(C)(C)C ; COC(=O)c1cc(F)cc(CN)c1 ; COC(=O)c1cc(F)cc(CNC(=S)NC(=O)OC(C)(C)C)c1]

To an ice cold solution of N,N-bisboc-thiourea (335.8 g, 1.21 mol) in THF (4.5 L) was added NaH (35 g) in a 12 L 3-neck RBF. After 1 hr at this temperature TFAA was added over 30 min, keeping the temperature close to 3° C. After stirring for an additional 1 hr a solution of methyl 3-(aminomethyl)-5-fluorobenzoate (133.7 g, 0.72 mol) in THF (0.3 L) was added over 30 min, keeping the temperature at 3° C. After stirring for 90 minutes the reaction mixture was poured into 18 L cold water. Then brine (2 L) was added to this solution and the resulting solution was extracted with 8 L EtOAc and, subsequently, 4 L EtOAc. The combined organic layer was dried over MgSO₄, filtered, and concentrated to dryness. The residue was further azeotroped with hexane 4 L, and solidified upon cooling. The solid was filtered, and washed with cold pentane. The solid residue was triturated with 40/60 isopropanol:pentane, chilled and filtered. The solid was further washed with 40/60 isopropanol:pentane (cold) 3×200 ml, filtered and dried under vacuum to give the desired product as a solid (230 g).

### Preparation of Compounds IX.

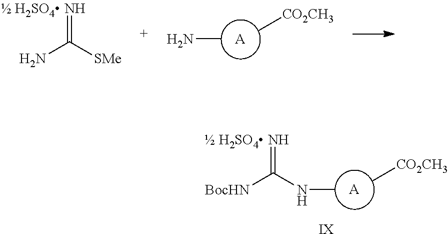

Compounds IX were generally prepared in the following manner. A solution of S-methylisothiourea sulfate and requisite amine were heated in water to afford compounds IX.

An example of the preparation of compound IX is shown below in Scheme 5

### Scheme 5:

1-(2,4-dimethoxybenzyl)Guanidine

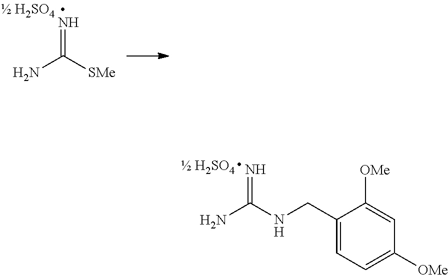 [SMILES: CSC(=N)N ; COc1ccc(CNC(=N)N)c(OC)c1]

The mixture of S-methylisothiourea sulfate (6.95 g, 50 mmol) and 2,4-dimethoxybenzylamine (8.36 g, 50 mmol) in 10 mL water was heated at 105° C. for 2 h. After this period the reaction mixture was cooled to room temperature, filtered, and rinsed with water. The filter cake was suspended in 40 mL water and was heated to boiling. The suspension was cooled to room temperature, filtered, rinsed with water and a 9:1 mixture of Et₂O and iPrOH. The crystalline product was dried in vacuo to provide 10.2 g of 1-(2,4-dimethoxybenzyl)guanidine.

### Preparation of Compound X.

An example of the preparation of compound X is shown below in Scheme 6.

### Scheme 6:

### Compound X

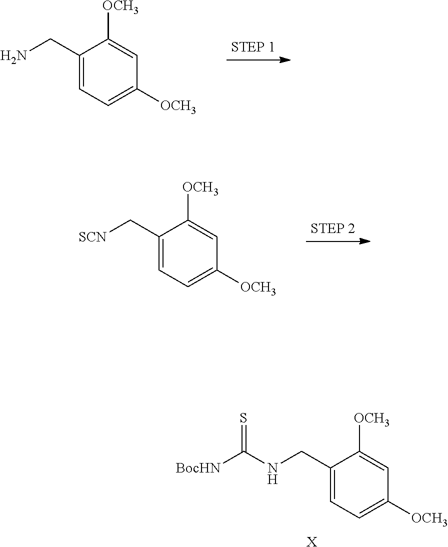

### Scheme 6, Step 1

1-(Isothiocyanatomethyl)-2,4-dimethoxybenzene

To a solution of 2,4-dimethoxybenzylamine (6.6 mL, 43.5 mmol) in DCM (85 mL) was added saturated aqueous sodium bicarbonate solution (85 mL) and the mixture was stirred vigorously at RT for 15 min. Stirring was stopped then thiophosgene (6.6 mL, 87 mmol) was added via syringe to the bottom layer. The mixture was stirred at RT for 90 min then the aqueous layer was separated and the organic layer was washed with brine, dried over Na₂SO₄ and concentrated in vacuo twice from DCM to give 9.1 g of 1-(isothiocyanatomethyl)-2,4-dimethoxybenzene as yellow oil.

### Scheme 6, Step 2

### Compound X

To a suspension of 60% sodium hydride in hexanes (3.4 g, 85 mmol) in anhydrous THF (100 mL) at 0° C. was added tert-butylcarbamate (7.4 g, 63 mmol) and the mixture was stirred for 15 min. A solution of 1-(isothiocyanatomethyl)-2,4-dimethoxybenzene (9.1 g, 43.5 mmol) in anhydrous THF (50 mL) was then added over 15 min and the reaction was allowed to warm up to RT and stirred overnight. The final mixture was quenched with water and 10% aqueous phosphoric acid until neutral pH, extracted with EtOAc, dried over Na₂SO₄ and concentrated in vacuo. The residue was purified by chromatography over silica gel (eluting with hexanes/EtOAc 100:0 to 80:20) to afford 10.61 g of compound X as a solid.

### Preparation of Compounds XIII.

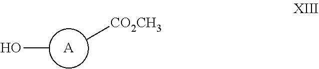

Precursors include, but are not limited to, requisite aldehydes, carboxylic esters, or carboxylic acids which may be treated with reducing reagents to afford the corresponding alcohols. Alternatively, requisite aldehydes may be treated with organometalic reagents to afford the corresponding secondary alcohols. Examples of the preparation of compounds XIII are shown below in Schemes 7 and 8.

### Scheme 7:

Methyl 5-(hydroxymethyl)thiophene-3-carboxylate

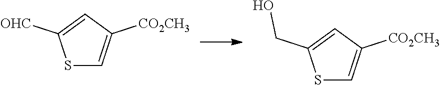 [SMILES: COC(=O)c1csc(C=O)c1 ; COC(=O)c1csc(CO)c1]

To a solution of methyl 5-formylthiophene-3-carboxylate (1.01 g, 5.93 mmol) in MEOH (60 mL) held at 0° C. was added sodium borohydride (0.45 g, 11.9 mmol). After the bubbling ceased, the reaction was allowed to warm to room temperature and stirred an additional 30 min. The mixture was extracted with EtOAc (200 mL) and brine (200 mL). The organic portion was collected, dried over sodium sulfate, filtered, and concentrated in vacuo to afford methyl 5-(hydroxymethyl)thiophene-3-carboxylate. (0.89 g) ¹H NMR (400 MHz, CDCl₃) δ8.04 (d, 1H), 7.40 (m, 1H), 4.82 (s, 2H), 3.85 (s, 3H).

### Scheme 8:

Methyl 4-(hydroxymethyl)-6-methylpicolinate

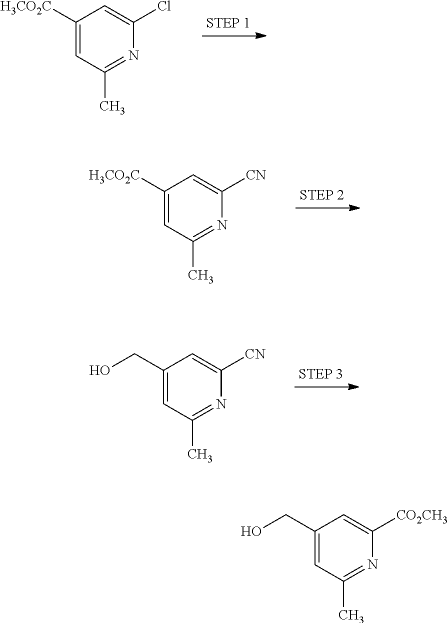

### Scheme 8, Step 1:

Methyl 2-cyano-6-methylisonicotinate

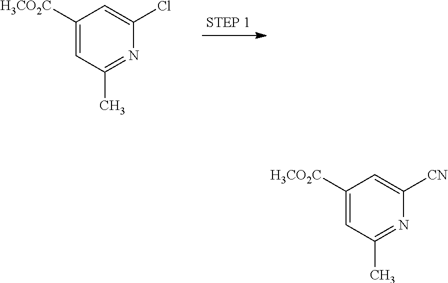

To a degassed solution of methyl 2-chloro-6-methylisonicotinate (2.03 g, 10.9 mmol) in DMF (20 mL) was added tetrakis(triphenylphosphine)palladium(0) (1.18 g, 1.02 mmol) and zinc cyanide (1.14 g, 9.71 mmol). This mixture was heated to 80° C. while stirring for 18 h under an atmosphere of nitrogen. The mixture was cooled to room temperature, then extracted with EtOAc (100 mL) and sat'd NH₄OH _{(aq)} solution (2×100 mL) then brine (2×100 mL). The combined organic extracts were dried over sodium sulfate, filtered, and concentrated in vacuo. The resultant residue was purified by silica gel chromatography eluting with 0 to 30% EtOAc in Hexanes to afford methyl 2-cyano-6-methylisonicotinate (1.29 g). Mass Spec. (ESI+) m/z=177.2 (M+H⁺).

### Scheme 8, Step 2:

Methyl 4-(hydroxymethyl)-6-methylpicolinonitrile

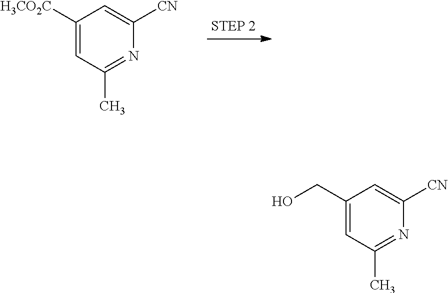

A solution of methyl 2-cyano-6-methylisonicotinate (602 mg, 3.42 mmol) in THF (5 mL) was cooled to 0° C. while stirring. To this cooled solution was added sodium borohydride (388 mg, 10.25 mmol). Then, to this solution was added MEOH (12 mL) and it was allowed to continue stirring at 0° C. for 90 min. The solution was warmed to room temperature and stirred an additional 15 min. The reaction mixture was quenched with a 1 N HCl (aq) solution to adjust the pH to ˜7. The mixture was extracted with EtOAc (50 mL) and brine (50 mL) and the combined organic portions were dried over sodium sulfate, filtered, and concentrated in vacuo to afford 4-(hydroxymethyl)-6-methylpicolinonitrile (0.50 g). ¹H NMR (400 MHz, CDCl₃) δ7.52 (s, 1H), 7.38 (s, 1H), 4.75 (s, 2H), 2.56 (s, 3H).

### Scheme 8, Step 3:

Methyl 4-(hydroxymethyl)-6-methylpicolinate

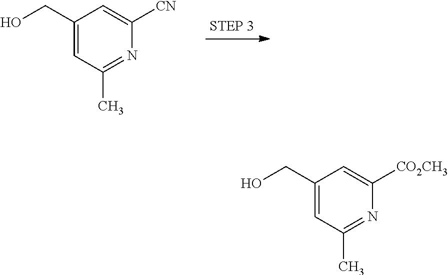

A solution of 4-(hydroxymethyl)-6-methylpicolinonitrile (0.50 g, 3.4 mmol) in water (3 mL) and conc. sulfuric acid (2 mL) was heated to 135° C. while stirring for 18 h. After this time the reaction was cooled to 95° C. and MEOH (8 mL) was added to the mixture and was allowed to stir at 95° C. for an additional 1 h. The reaction was allowed to cool to room temperature. Then the contents of the reaction mixture were added directly over ice. Water (100 mL) and EtOAc (100 mL) were added to this mixture. Solid sodium bicarbonate was added to the solution to adjust to pH ˜8. The aqueous layer was extracted with EtOAc (2×100 mL). The combined organic portions were washed with brine (100 mL), dried over sodium sulfate, and concentrated to afford methyl 4-(hydroxymethyl)-6-methylpicolinate (170.9 mg).¹H NMR (400 MHz, CDCl₃) δ7.90 (s, 1H), 7.36 (s, 1H), 4.76 (s, 2H), 3.97 (s, 3H), 2.63 (s, 3H). Mass Spec. (ESI+) m/z=182.2 (M+H⁺).

The compounds shown in Table 1 were made by following procedures analogous to the schemes and Examples herein.

TABLE 1

| | | | Exact |
|---|---|---|---|
| Compd | | | Mass |
| # | Structure | IUPAC Name | [[M + H]+ |
| 1 | 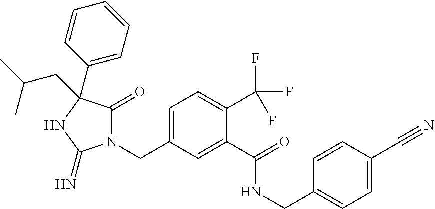 [SMILES: CC(C)CC1(c2ccccc2)NC(=N)N(Cc2ccc(C(F)(F)F)c(C(=O)NCc3ccc(C#N)cc3)c2)C1=O] | N-(4-cyanobenzyl)-5-{[2-imino- 4-(2-methylpropyl)-5-oxo-4- phenylimidazolidin-1- yl]methyl}-2- (trifluoromethyl)benzamide | 548.2 |
| 2 | 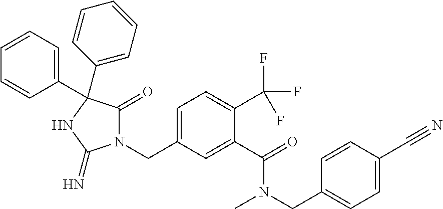 [SMILES: CN(Cc1ccc(C#N)cc1)C(=O)c1cc(CN2C(=N)NC(c3ccccc3)(c3ccccc3)C2=O)ccc1C(F)(F)F] | N-(4-cyanobenzyl)-5-[(2-imino- 5-oxo-4,4-diphenylimidazolidin- 1-yl)methyl]-N-methyl-2- (trifluoromethyl)benzamide | 582.2 |
| 3 | 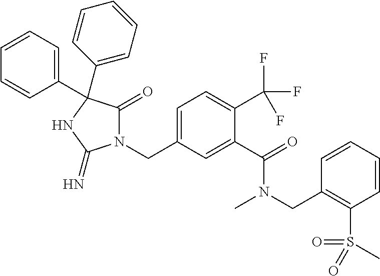 [SMILES: CN(Cc1ccccc1S(C)(=O)=O)C(=O)c1cc(CN2C(=N)NC(c3ccccc3)(c3ccccc3)C2=O)ccc1C(F)(F)F] | 5-[(2-imino-5-oxo-4,4- diphenylimidazolidin-1- yl)methyl]-N-methyl-N-[2- (methylsulfonyl)benzyl]-2- (trifluoromethyl)benzamide | 635.2 |
| 4 | 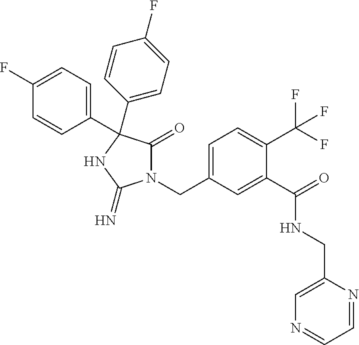 [SMILES: N=C1NC(c2ccc(F)cc2)(c2ccc(F)cc2)C(=O)N1Cc1ccc(C(F)(F)F)c(C(=O)NCc2cnccn2)c1] | 5-{[4,4-bis(4-fluorophenyl)-2- imino-5-oxoimidazolidin-1- yl]methyl}-N-(pyrazin-2- ylmethyl)-2- (trifluoromethyl)benzamide | 581.2 |
| 5 | 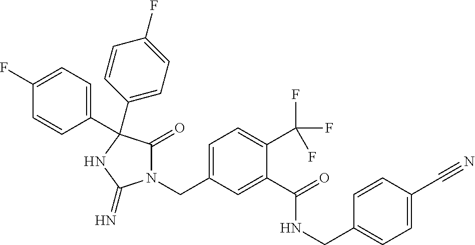 [SMILES: N#Cc1ccc(CNC(=O)c2cc(CN3C(=N)NC(c4ccc(F)cc4)(c4ccc(F)cc4)C3=O)ccc2C(F)(F)F)cc1] | 5-{[4,4-bis(4-fluorophenyl)-2- imino-5-oxoimidazolidin-1- yl]methyl}-N-(4-cyanobenzyl)- 2-(trifluoromethyl)benzamide | 604.2 |
| 6 | 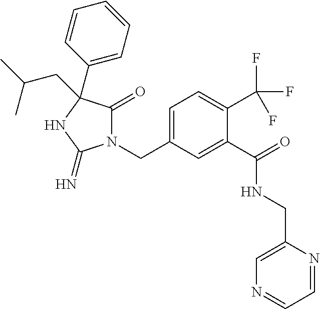 [SMILES: CC(C)CC1(c2ccccc2)NC(=N)N(Cc2ccc(C(F)(F)F)c(C(=O)NCc3cnccn3)c2)C1=O] | 5-{[2-imino-4-(2-methylpropyl)- 5-oxo-4-phenylimidazolidin-1- yl]methyl}-N-(pyrazin-2- ylmethyl)-2- (trifluoromethyl)benzamide | 525.2 |
| 7 | 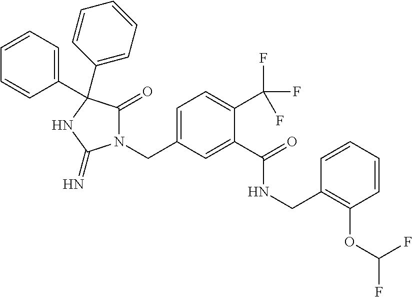 [SMILES: N=C1NC(c2ccccc2)(c2ccccc2)C(=O)N1Cc1ccc(C(F)(F)F)c(C(=O)NCc2ccccc2OC(F)F)c1] | N-[2-(difluoromethoxy)benzyl]- 5-[(2-imino-5-oxo-4,4- diphenylimidazolidin-1- yl)methyl]-2- (trifluoromethyl)benzamide | 609.2 |
| 8 | 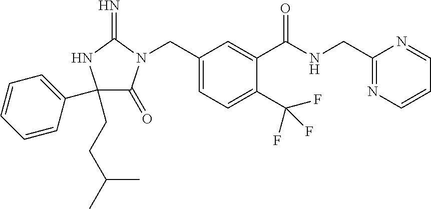 [SMILES: CC(C)CCC1(c2ccccc2)NC(=N)N(Cc2ccc(C(F)(F)F)c(C(=O)NCc3ncccn3)c2)C1=O] | 5-{[2-imino-4-(3-methylbutyl)- 5-oxo-4-phenylimidazolidin-1- yl]methyl}-N-(pyrimidin-2- ylmethyl)-2- (trifluoromethyl)benzamide | 539.2 |
| 9 | 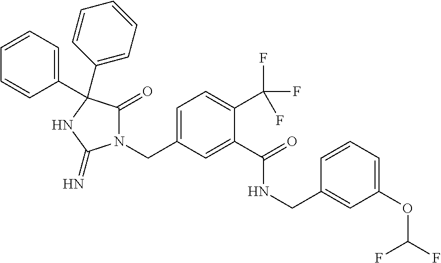 [SMILES: N=C1NC(c2ccccc2)(c2ccccc2)C(=O)N1Cc1ccc(C(F)(F)F)c(C(=O)NCc2cccc(OC(F)F)c2)c1] | N-[3-(difluoromethoxy)benzyl]- 5-[(2-imino-5-oxo-4,4- diphenylimidazolidin-1- yl)methyl]-2- (trifluoromethyl)benzamide | 609.2 |
| 10 | 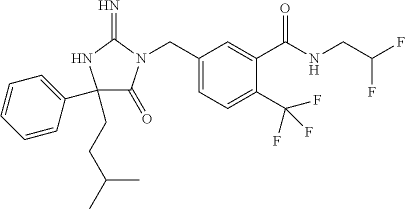 [SMILES: CC(C)CCC1(c2ccccc2)NC(=N)N(Cc2ccc(C(F)(F)F)c(C(=O)NCC(F)F)c2)C1=O] | N-(2,2-difluoroethyl)-5-{[2- imino-4-(3-methylbutyl)-5-oxo- 4-phenylimidazolidin-1- yl]methyl}-2- (trifluoromethyl)benzamide | 511.2 |
| 11 | 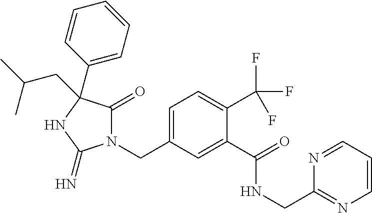 [SMILES: CC(C)CC1(c2ccccc2)NC(=N)N(Cc2ccc(C(F)(F)F)c(C(=O)NCc3ncccn3)c2)C1=O] | 5-{[2-imino-4-(2-methylpropyl)- 5-oxo-4-phenylimidazolidin-1- yl]methyl}-N-(pyrimidin-2- ylmethyl)-2-(trifluoromethyl)- benzamide | 525.2 |
| 12 | 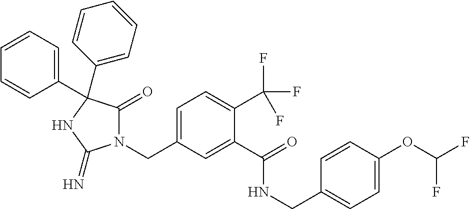 [SMILES: N=C1NC(c2ccccc2)(c2ccccc2)C(=O)N1Cc1ccc(C(F)(F)F)c(C(=O)NCc2ccc(OC(F)F)cc2)c1] | N-[4-(difluoromethoxy)benzyl]- 5-[(2-imino-5-oxo-4,4- diphenylimidazolidin-1- yl)methyl]-2- (trifluoromethyl)benzamide | 609.2 |
| 13 | 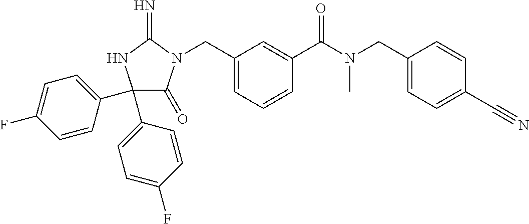 [SMILES: CN(Cc1ccc(C#N)cc1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(4-cyanobenzyl)- N-methylbenzamide | 550.2 |
| 14 | 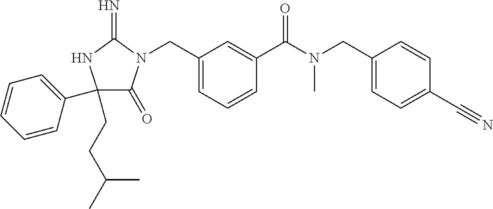 [SMILES: CC(C)CCC1(c2ccccc2)NC(=N)N(Cc2cccc(C(=O)N(C)Cc3ccc(C#N)cc3)c2)C1=O] | N-(4-cyanobenzyl)-3-{[2-imino- 4-(3-methylbutyl)-5-oxo-4- phenylimidazolidin-1- yl]methyl}-N-methylbenzamide | 508.3 |
| 15 | 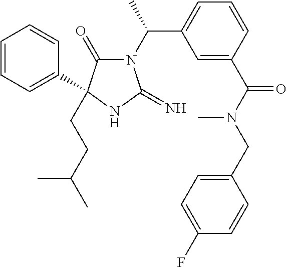 [SMILES: CC(C)CC[C@]1(c2ccccc2)NC(=N)N([C@H](C)c2cccc(C(=O)N(C)Cc3ccc(F)cc3)c2)C1=O] | N-(4-fluorobenzyl)-3-{(1R)-1- [2-imino-4-(3-methylbutyl)-5- oxo-4-phenylimidazolidin-1- yl]ethyl}-N-methylbenzamide | 515.3 |
| 16 | 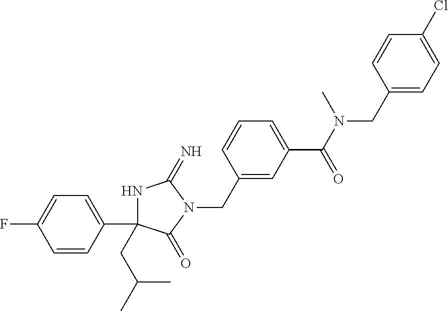 [SMILES: CC(C)CC1(c2ccc(F)cc2)NC(=N)N(Cc2cccc(C(=O)N(C)Cc3ccc(Cl)cc3)c2)C1=O] | N-(4-chlorobenzyl)-3-{[4-(4- fluorophenyl)-2-imino-4-(2- methylpropyl)-5- oxoimidazolidin-1-yl]methyl}- N-methylbenzamide | 521.2 |
| 17 | 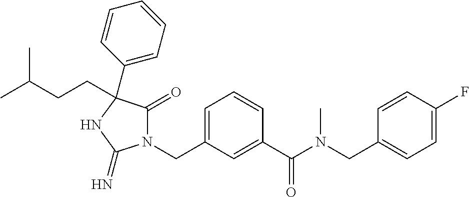 [SMILES: CC(C)CCC1(c2ccccc2)NC(=N)N(Cc2cccc(C(=O)N(C)Cc3ccc(F)cc3)c2)C1=O] | N-(4-fluorobenzyl)-3-{[2-imino- 4-(3-methylbutyl)-5-oxo-4- phenylimidazolidin-1- yl]methyl}-N-methylbenzamide | 501.3 |
| 18 | 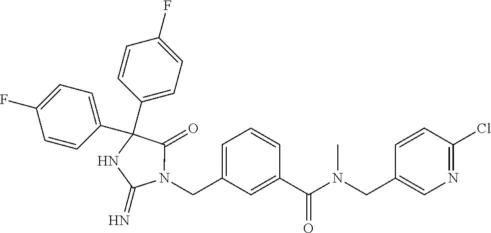 [SMILES: CN(Cc1ccc(Cl)nc1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-[(6-chloropyridin- 3-yl)methyl]-N- methylbenzamide | 560.2 |
| 19 | 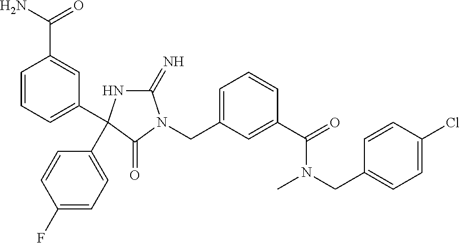 [SMILES: CN(Cc1ccc(Cl)cc1)C(=O)c1cccc(CN2C(=N)NC(c3ccc(F)cc3)(c3cccc(C(N)=O)c3)C2=O)c1] | 3-{[4-(3-carbamoylphenyl)-4-(4- fluorophenyl)-2-imino-5- oxoimidazolidin-1-yl]methyl}- N-(4-chlorobenzyl)-N- methylbenzamide | 584.2 |
| 20 | 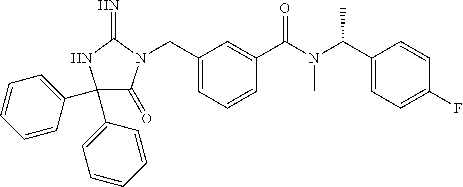 [SMILES: C[C@H](c1ccc(F)cc1)N(C)C(=O)c1cccc(CN2C(=N)NC(c3ccccc3)(c3ccccc3)C2=O)c1] | N-[(1R)-1-(4- fluorophenyl)ethyl]-3-[(2-imino- 5-oxo-4,4-diphenylimidazolidin- 1-yl)methyl]-N- methylbenzamide | 521.2 |
| 21 | 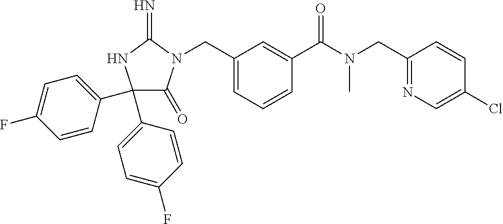 [SMILES: CN(Cc1ccc(Cl)cn1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-[(5-chloropyridin- 2-yl)methyl]-N- methylbenzamide | 560.2 |
| 22 | 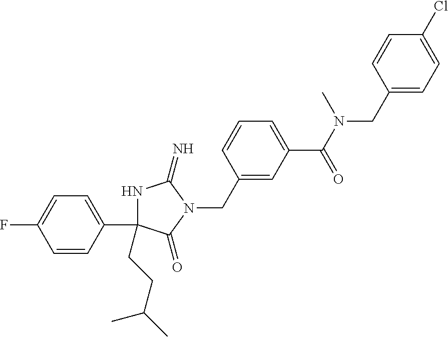 [SMILES: CC(C)CCC1(c2ccc(F)cc2)NC(=N)N(Cc2cccc(C(=O)N(C)Cc3ccc(Cl)cc3)c2)C1=O] | N-(4-chlorobenzyl)-3-{[4-(4- fluorophenyl)-2-imino-4-(3- methylbutyl)-5-oxoimidazolidin- 1-yl]methyl}-N- methylbenzamide | 535.2 |
| 23 | 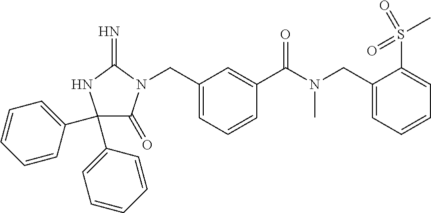 [SMILES: CN(Cc1ccccc1S(C)(=O)=O)C(=O)c1cccc(CN2C(=N)NC(c3ccccc3)(c3ccccc3)C2=O)c1] | 3-[(2-imino-5-oxo-4,4- diphenylimidazolidin-1- yl)methyl]-N-methyl-N-[2- (methylsulfonyl)- benzyl]benzamide | 567.2 |
| 24 | 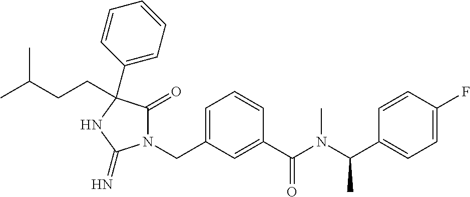 [SMILES: CC(C)CCC1(c2ccccc2)NC(=N)N(Cc2cccc(C(=O)N(C)[C@H](C)c3ccc(F)cc3)c2)C1=O] | N-[(1R)-1-(4- fluorophenyl)ethyl]-3-{[2- imino-4-(3-methylbutyl)-5-oxo- 4-phenylimidazolidin-1- yl]methyl}-N-methylbenzamide | 515.3 |
| 25 | 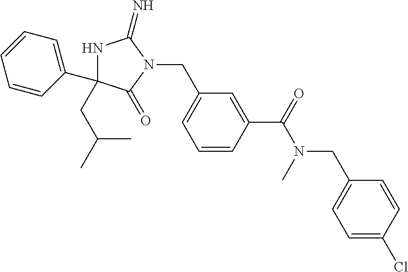 [SMILES: CC(C)CC1(c2ccccc2)NC(=N)N(Cc2cccc(C(=O)N(C)Cc3ccc(Cl)cc3)c2)C1=O] | N-(4-chlorobenzyl)-3-{[2-imino- 4-(2-methylpropyl)-5-oxo-4- phenylimidazolidin-1- yl]methyl}-N-methylbenzamide | 503.2 |
| 26 | 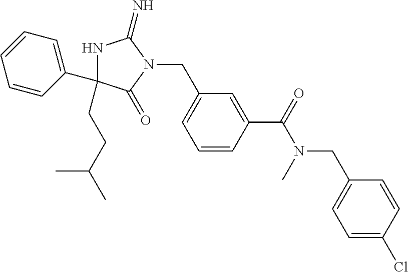 [SMILES: CC(C)CCC1(c2ccccc2)NC(=N)N(Cc2cccc(C(=O)N(C)Cc3ccc(Cl)cc3)c2)C1=O] | N-(4-chlorobenzyl)-3-{[2-imino- 4-(3-methylbutyl)-5-oxo-4- phenylimidazolidin-1- yl]methyl}-N-methylbenzamide | 517.2 |
| 27 | 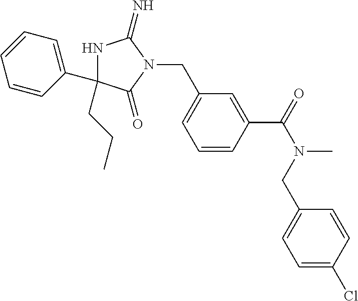 [SMILES: CCCC1(c2ccccc2)NC(=N)N(Cc2cccc(C(=O)N(C)Cc3ccc(Cl)cc3)c2)C1=O] | N-(4-chlorobenzyl)-3-[(2-imino- 5-oxo-4-phenyl-4- propylimidazolidin-1-yl)methyl]- N-methylbenzamide | 489.2 |
| 28 | 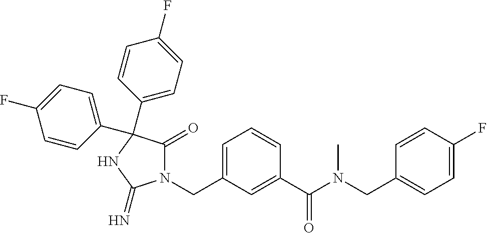 [SMILES: CN(Cc1ccc(F)cc1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(4-fluorobenzyl)-N- methylbenzamide | 543.2 |
| 29 | 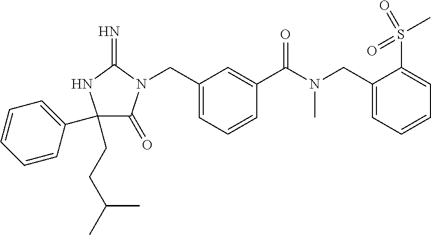 [SMILES: CC(C)CCC1(c2ccccc2)NC(=N)N(Cc2cccc(C(=O)N(C)Cc3ccccc3S(C)(=O)=O)c2)C1=O] | 3-{[2-imino-4-(3-methylbutyl)-5- oxo-4-phenylimidazolidin-1- yl]methyl}-N-methyl-N-[2- (methylsulfonyl)- benzyl]benzamide | 561.3 |
| 30 | 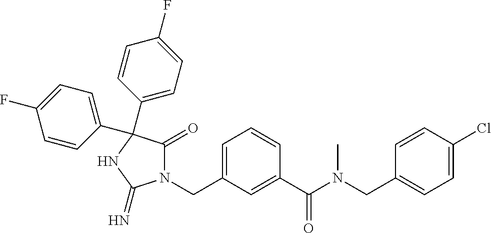 [SMILES: CN(Cc1ccc(Cl)cc1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(4-chlorobenzyl)-N- methylbenzamide | 559.2 |
| 31 | 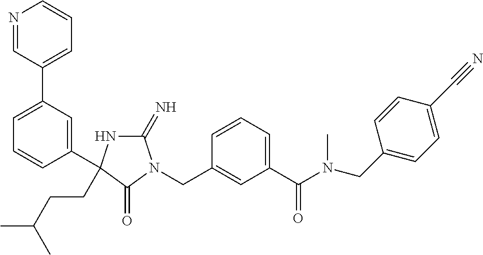 [SMILES: CC(C)CCC1(c2cccc(-c3cccnc3)c2)NC(=N)N(Cc2cccc(C(=O)N(C)Cc3ccc(C#N)cc3)c2)C1=O] | N-(4-cyanobenzyl)-3-{[2-imino- 4-(3-methylbutyl)-5-oxo-4-(3- pyridin-3-ylphenyl)imidazolidin- 1-yl]methyl}-N-methylbenzamide | 585.3 |
| 32 | 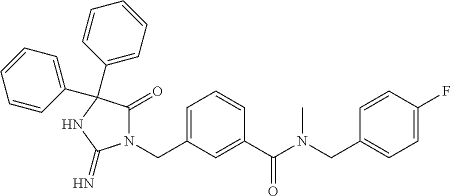 [SMILES: CN(Cc1ccc(F)cc1)C(=O)c1cccc(CN2C(=N)NC(c3ccccc3)(c3ccccc3)C2=O)c1] | N-(4-fluorobenzyl)-3-[(2-imino-5- oxo-4,4-diphenylimidazolidin-1- yl)methyl]-N-methylbenzamide | 507.2 |
| 33 | 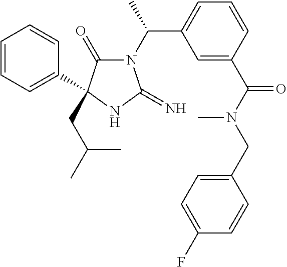 [SMILES: CC(C)C[C@]1(c2ccccc2)NC(=N)N([C@H](C)c2cccc(C(=O)N(C)Cc3ccc(F)cc3)c2)C1=O] | N-(4-fluorobenzyl)-3-((R)-1-((R)- 2-imino-4-isobutyl-5-oxo-4- phenylimidazolidin-1-yl)ethyl)-N- methylbenzamide | 501.3 |
| 34 | 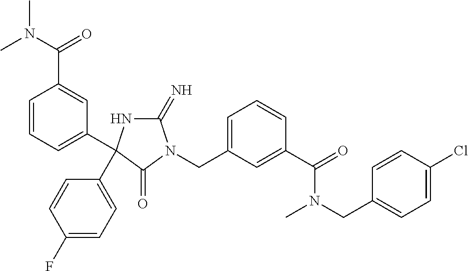 [SMILES: CN(C)C(=O)c1cccc(C2(c3ccc(F)cc3)NC(=N)N(Cc3cccc(C(=O)N(C)Cc4ccc(Cl)cc4)c3)C2=O)c1] | 3-[1-{3-[(4- chlorobenzyl)(methyl)- carbamoyl]benzyl}-4-(4- fluorophenyl)-2-imino-5- oxoimidazolidin-4-yl]-N,N- dimethylbenzamide | 612.2 |
| 35 | 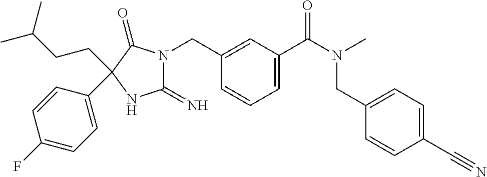 [SMILES: CC(C)CCC1(c2ccc(F)cc2)NC(=N)N(Cc2cccc(C(=O)N(C)Cc3ccc(C#N)cc3)c2)C1=O] | N-(4-cyanobenzyl)-3-{[4-(4- fluorophenyl)-2-imino-4-(3- methylbutyl)-5-oxoimidazolidin- 1-yl]methyl}-N- methylbenzamide | 526.3 |
| 36 | 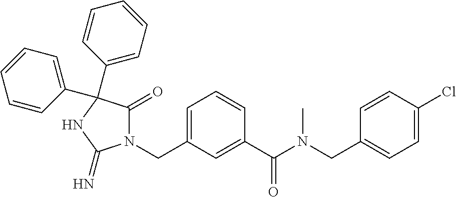 [SMILES: CN(Cc1ccc(Cl)cc1)C(=O)c1cccc(CN2C(=N)NC(c3ccccc3)(c3ccccc3)C2=O)c1] | N-(4-chlorobenzyl)-3-[(2-imino- 5-oxo-4,4-diphenylimidazolidin- 1-yl)methyl]-N-methylbenzamide | 523.2 |
| 37 | 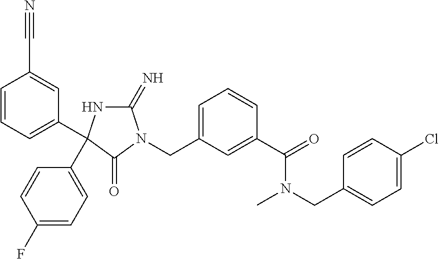 [SMILES: CN(Cc1ccc(Cl)cc1)C(=O)c1cccc(CN2C(=N)NC(c3ccc(F)cc3)(c3cccc(C#N)c3)C2=O)c1] | N-(4-chlorobenzyl)-3-{[4-(3- cyanophenyl)-4-(4-fluorophenyl)- 2-imino-5-oxoimidazolidin-1- yl]methyl}-N-methylbenzamide | 566.2 |
| 38 | 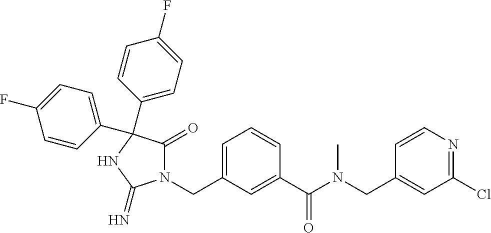 [SMILES: CN(Cc1ccnc(Cl)c1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-[(2-chloropyridin- 4-yl)methyl]-N-methylbenzamide | 560.2 |
| 39 | 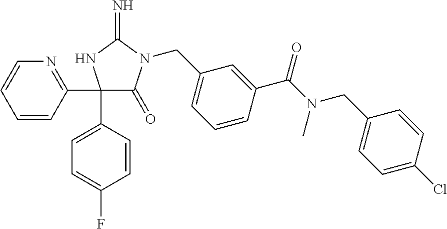 [SMILES: CN(Cc1ccc(Cl)cc1)C(=O)c1cccc(CN2C(=N)NC(c3ccc(F)cc3)(c3ccccn3)C2=O)c1] | N-(4-chlorobenzyl)-3-{[4-(4- fluorophenyl)-2-imino-5-oxo-4- pyridin-2-ylimidazolidin-1- yl]methyl}-N-methylbenzamide | 542.2 |
| 40 | 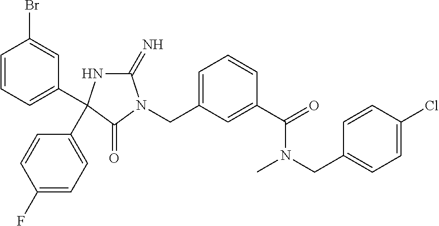 [SMILES: CN(Cc1ccc(Cl)cc1)C(=O)c1cccc(CN2C(=N)NC(c3ccc(F)cc3)(c3cccc(Br)c3)C2=O)c1] | 3-{[4-(3-bromophenyl)-4-(4- fluorophenyl)-2-imino-5- oxoimidazolidin-1-yl]methyl}-N- (4-chlorobenzyl)-N- methylbenzamide | 619.1 |
| 41 | 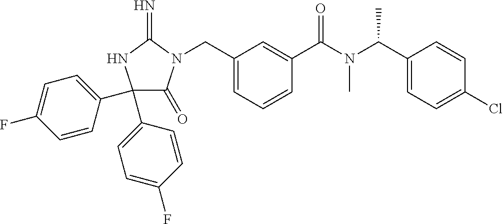 [SMILES: C[C@H](c1ccc(Cl)cc1)N(C)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-[(1R)-1-(4- chlorophenyl)ethyl]-N- methylbenzamide | 573.2 |
| 42 | 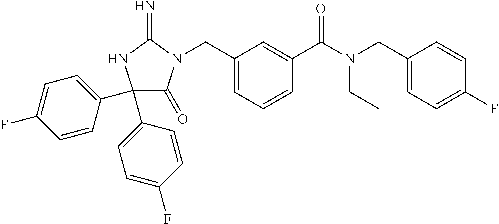 [SMILES: CCN(Cc1ccc(F)cc1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-ethyl-N-(4- fluorobenzyl)benzamide | 557.2 |
| 43 | 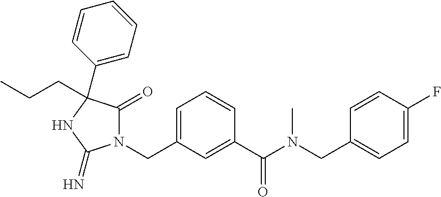 [SMILES: CCCC1(c2ccccc2)NC(=N)N(Cc2cccc(C(=O)N(C)Cc3ccc(F)cc3)c2)C1=O] | N-(4-fluorobenzyl)-3-[(2-imino- 5-oxo-4-phenyl-4- propylimidazolidin-1-yl)methyl]- N-methylbenzamide | 473.2 |
| 44 | 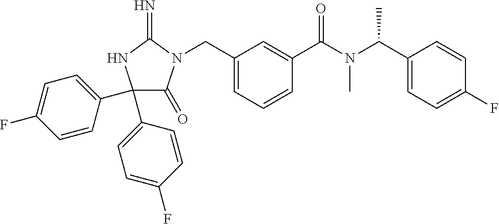 [SMILES: C[C@H](c1ccc(F)cc1)N(C)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-[(1R)-1-(4- fluorophenyl)ethyl]-N- methylbenzamide | 557.2 |
| 45 | 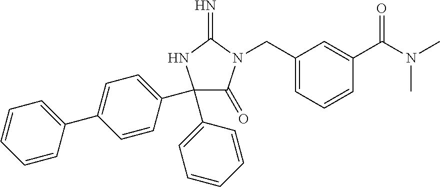 [SMILES: CN(C)C(=O)c1cccc(CN2C(=N)NC(c3ccccc3)(c3ccc(-c4ccccc4)cc3)C2=O)c1] | 3-[(4-biphenyl-4-yl-2-imino-5- oxo-4-phenylimidazolidin-1- yl)methyl]-N,N- dimethylbenzamide | 489.2 |
| 46 | 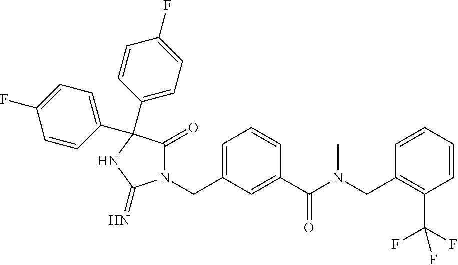 [SMILES: CN(Cc1ccccc1C(F)(F)F)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-methyl-N-[2- (trifluoromethyl)- benzyl]benzamide | 593.2 |
| 47 | 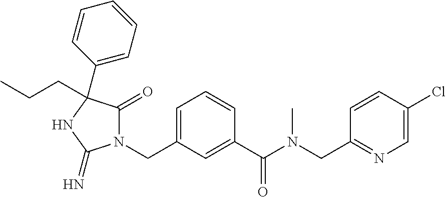 [SMILES: CCCC1(c2ccccc2)NC(=N)N(Cc2cccc(C(=O)N(C)Cc3ccc(Cl)cn3)c2)C1=O] | N-[(5-chloropyridin-2- yl)methyl]-3-[(2-imino-5-oxo-4- phenyl-4-propylimidazolidin-1- yl)methyl]-N-methylbenzamide | 490.2 |
| 48 | 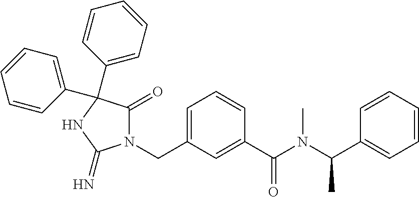 [SMILES: C[C@H](c1ccccc1)N(C)C(=O)c1cccc(CN2C(=N)NC(c3ccccc3)(c3ccccc3)C2=O)c1] | 3-[(2-imino-5-oxo-4,4- diphenylimidazolidin-1-yl)methyl]- N-methyl-N-[(1R)-1- phenylethyl]benzamide | 503.2 |
| 49 | 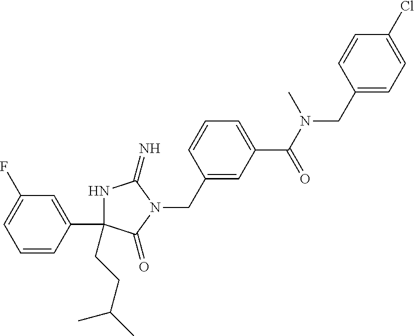 [SMILES: CC(C)CCC1(c2cccc(F)c2)NC(=N)N(Cc2cccc(C(=O)N(C)Cc3ccc(Cl)cc3)c2)C1=O] | N-(4-chlorobenzyl)-3-{[4-(3- fluorophenyl)-2-imino-4-(3- methylbutyl)-5-oxoimidazolidin-1- yl]methyl}-N-methylbenzamide | 535.2 |
| 50 | 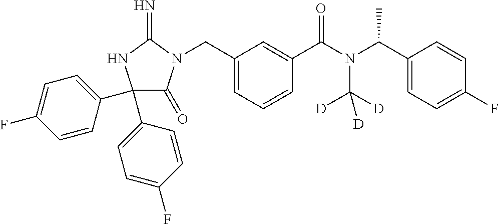 [SMILES: [2H]C([2H])([2H])N(C(=O)c1cccc(CN2C(=N)NC(c3ccc(F)cc3)(c3ccc(F)cc3)C2=O)c1)[C@H](C)c1ccc(F)cc1] | 3-{[4,4-bis(4-fluorophenyl)-2-imino- 5-oxoimidazolidin-1-yl]methyl}-N- [(1R)-1-(4-fluorophenyl)ethyl]- N(~2~H_3_)methylbenzamide | 560.2 |
| 51 | 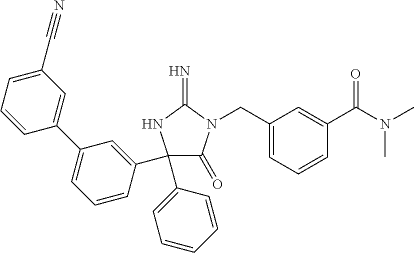 [SMILES: CN(C)C(=O)c1cccc(CN2C(=N)NC(c3ccccc3)(c3cccc(-c4cccc(C#N)c4)c3)C2=O)c1] | 3-{[4-(3'-cyanobiphenyl-3-yl)-2- imino-5-oxo-4-phenylimidazolidin- 1-yl]methyl}-N,N- dimethylbenzamide | 514.2 |
| 52 | 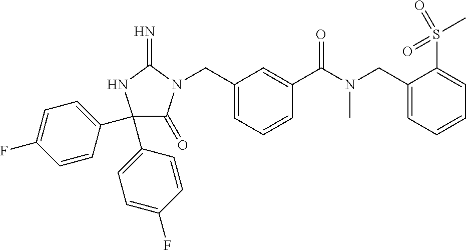 [SMILES: CN(Cc1ccccc1S(C)(=O)=O)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-methyl-N-[2- (methylsulfonyl)-benzyl]benzamide | 603.2 |
| 53 | 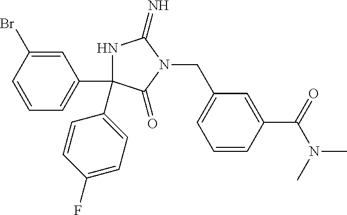 [SMILES: CN(C)C(=O)c1cccc(CN2C(=N)NC(c3ccc(F)cc3)(c3cccc(Br)c3)C2=O)c1] | 3-{[4-(3-bromophenyl)-4-(4- fluorophenyl)-2-imino-5- oxoimidazolidin-1-yl]methyl}-N,N- dimethylbenzamide | 509.1 |
| 54 | 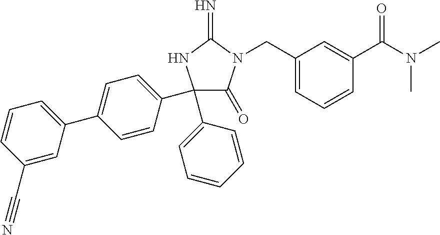 [SMILES: CN(C)C(=O)c1cccc(CN2C(=N)NC(c3ccccc3)(c3ccc(-c4cccc(C#N)c4)cc3)C2=O)c1] | 3-{[4-(3'-cyanobiphenyl-4-yl)-2- imino-5-oxo-4-phenylimidazolidin- 1-yl]methyl}-N,N- dimethylbenzamide | 514.2 |
| 55 | 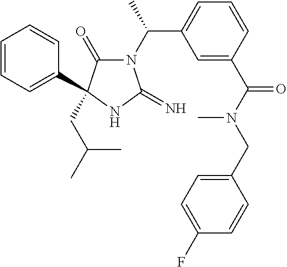 [SMILES: CC(C)C[C@@]1(c2ccccc2)NC(=N)N([C@H](C)c2cccc(C(=O)N(C)Cc3ccc(F)cc3)c2)C1=O] | N-(4-fluorobenzyl)-3-((R)-1-((S)-2- imino-4-isobutyl-5-oxo-4- phenylimidazolidin-1-yl)ethyl)-N- methylbenzamide | 501.3 |
| 56 | 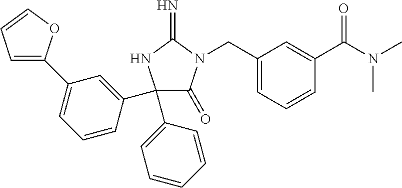 [SMILES: CN(C)C(=O)c1cccc(CN2C(=N)NC(c3ccccc3)(c3cccc(-c4ccco4)c3)C2=O)c1] | 3-{[4-(3-furan-2-ylphenyl)-2-imino- 5-oxo-4-phenylimidazolidin-1- yl]methyl}-N,N-dimethylbenzamide | 479.2 |
| 57 | 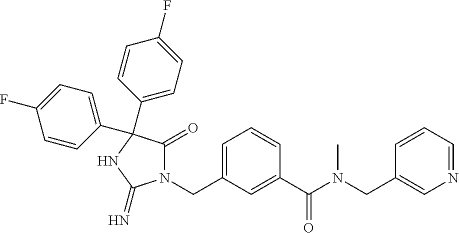 [SMILES: CN(Cc1cccnc1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-methyl-N-(pyridin-3- ylmethyl)benzamide | 526.2 |
| 58 | 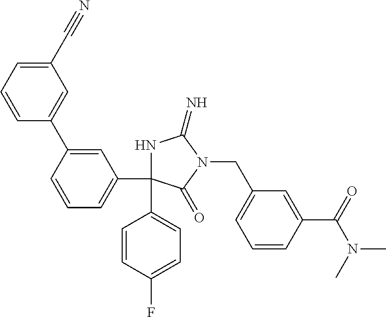 [SMILES: CN(C)C(=O)c1cccc(CN2C(=N)NC(c3ccc(F)cc3)(c3cccc(-c4cccc(C#N)c4)c3)C2=O)c1] | 3-{[4-(3'-cyanobiphenyl-3-yl)-4-(4- fluorophenyl)-2-imino-5- oxoimidazolidin-1-yl]methyl}-N,N- dimethylbenzamide | 532.2 |
| 59 | 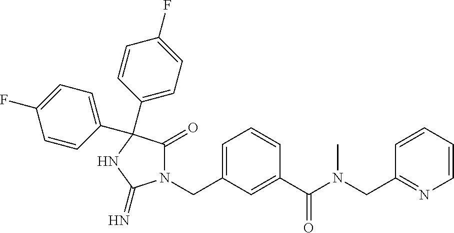 [SMILES: CN(Cc1ccccn1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-methyl-N-(pyridin-2- ylmethyl)benzamide | 526.2 |
| 60 | 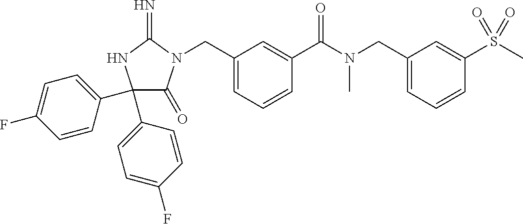 [SMILES: CN(Cc1cccc(S(C)(=O)=O)c1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-methyl-N-[3- (methylsulfonyl)benzyl]benzamide | 603.2 |
| 61 | 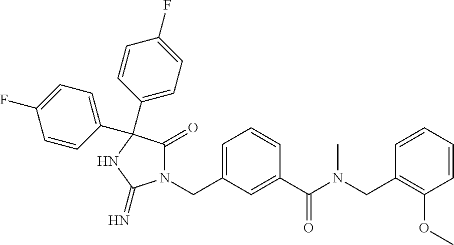 [SMILES: COc1ccccc1CN(C)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(2-methoxybenzyl)- N-methylbenzamide | 555.2 |
| 62 | 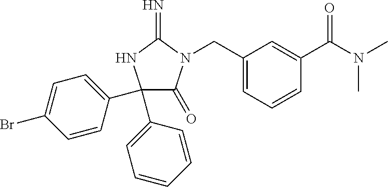 [SMILES: CN(C)C(=O)c1cccc(CN2C(=N)NC(c3ccccc3)(c3ccc(Br)cc3)C2=O)c1] | 3-{[4-(4-bromophenyl)-2-imino-5- oxo-4-phenylimidazolidin-1- yl]methyl}-N,N-dimethylbenzamide | 491.1 |
| 63 | 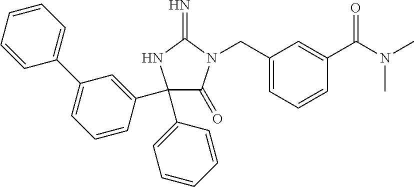 [SMILES: CN(C)C(=O)c1cccc(CN2C(=N)NC(c3ccccc3)(c3cccc(-c4ccccc4)c3)C2=O)c1] | 3-[(4-biphenyl-3-yl-2-imino-5-oxo- 4-phenylimidazolidin-1-yl)methyl]- N,N-dimethylbenzamide | 489.2 |
| 64 | 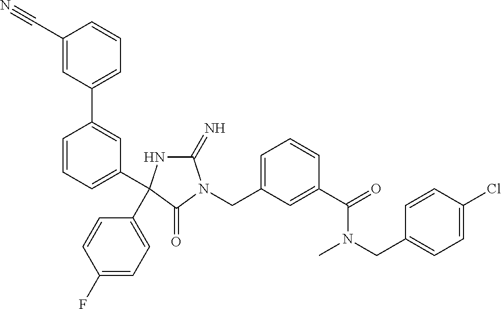 [SMILES: CN(Cc1ccc(Cl)cc1)C(=O)c1cccc(CN2C(=N)NC(c3ccc(F)cc3)(c3cccc(-c4cccc(C#N)c4)c3)C2=O)c1] | N-(4-chlorobenzyl)-3-{[4-(3'- cyanobiphenyl-3-yl)-4-(4- fluorophenyl)-2-imino-5- oxoimidazolidin-1-yl]methyl}-N- methylbenzamide | 642.2 |
| 65 | 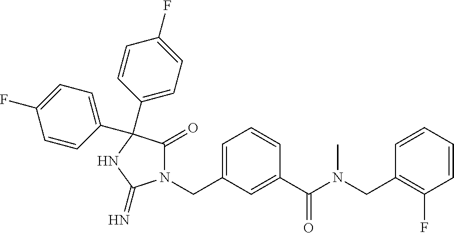 [SMILES: CN(Cc1ccccc1F)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(2-fluorobenzyl)-N- methylbenzamide | 543.2 |
| 66 | 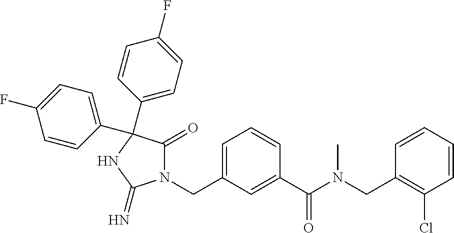 [SMILES: CN(Cc1ccccc1Cl)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(2-chlorobenzyl)-N- methylbenzamide | 559.2 |
| 67 | 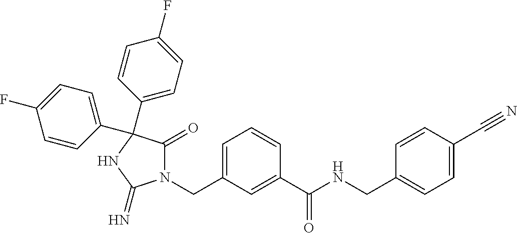 [SMILES: N#Cc1ccc(CNC(=O)c2cccc(CN3C(=N)NC(c4ccc(F)cc4)(c4ccc(F)cc4)C3=O)c2)cc1] | 3-{[4,4-bis(4-fluorophenyl)-2- imino-5-oxoimidazolidin-1- yl]methyl}-N-(4- cyanobenzyl)benzamide | 536.2 |
| 68 | 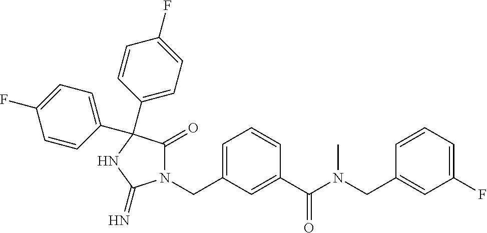 [SMILES: CN(Cc1cccc(F)c1)C(=O)c1cccc(CN2C(=N)NC(c3ccc(F)cc3)(c3ccc(F)cc3)C2=O)c1] | 3-{[4,4-bis(4-fluorophenyl)-2- imino-5-oxoimidazolidin-1- yl]methyl}-N-(3-fluorobenzyl)-N- methylbenzamide | 543.2 |
| 69 | 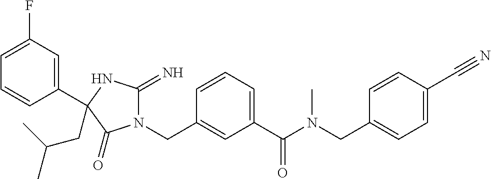 [SMILES: CC(C)CC1(c2cccc(F)c2)NC(=N)N(Cc2cccc(C(=O)N(C)Cc3ccc(C#N)cc3)c2)C1=O] | N-(4-cyanobenzyl)-3-{[4-(3- fluorophenyl)-2-imino-4-(2- methylpropyl)-5-oxoimidazolidin- 1-yl]methyl}-N-methylbenzamide | 512.2 |
| 70 | 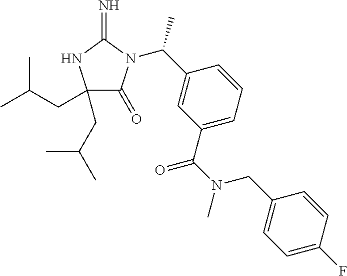 [SMILES: CC(C)CC1(CC(C)C)NC(=N)N([C@H](C)c2cccc(C(=O)N(C)Cc3ccc(F)cc3)c2)C1=O] | N-(4-fluorobenzyl)-3-{(1R)-1-[2- imino-4,4-bis(2-methylpropyl)-5_ oxoimidazolidin-1-yl]ethyl}-N- methylbenzamide | 481.3 |
| 71 | 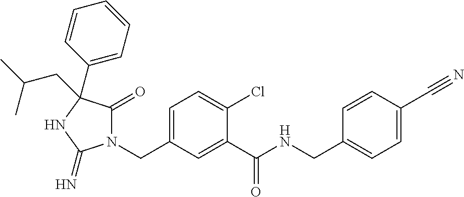 [SMILES: CC(C)CC1(c2ccccc2)NC(=N)N(Cc2ccc(Cl)c(C(=O)NCc3ccc(C#N)cc3)c2)C1=O] | 2-chloro-N-(4-cyanobenzyl)-5-{[2- imino-4-(2-methylpropyl)-5-oxo-4- phenylimidazolidin-1- yl]methyl}benzamide | 514.2 |
| 72 | 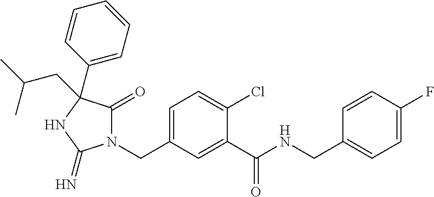 [SMILES: CC(C)CC1(c2ccccc2)NC(=N)N(Cc2ccc(Cl)c(C(=O)NCc3ccc(F)cc3)c2)C1=O] | 2-chloro-N-(4-fluorobenzyl)-5-{[2- imino-4-(2-methylpropyl)-5-oxo-4- phenylimidazolidin-1- yl]methyl}benzamide | 507.2 |
| 73 | 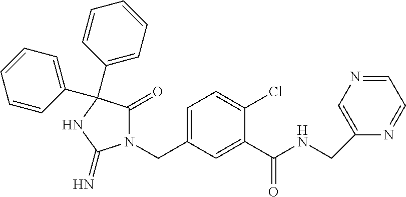 [SMILES: N=C1NC(c2ccccc2)(c2ccccc2)C(=O)N1Cc1ccc(Cl)c(C(=O)NCc2cnccn2)c1] | 2-chloro-5-[(2-imino-5-oxo-4,4- diphenylimidazolidin-1-yl)methyl]- N-(pyrazin-2-ylmethyl)benzamide | 511.2 |
| 74 | 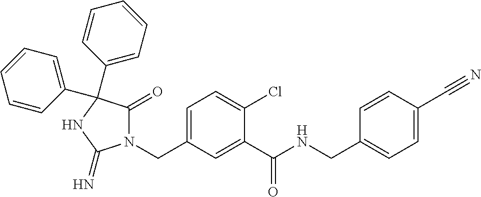 [SMILES: N#Cc1ccc(CNC(=O)c2cc(CN3C(=N)NC(c4ccccc4)(c4ccccc4)C3=O)ccc2Cl)cc1] | 2-chloro-N-(4-cyanobenzyl)-5-[(2- imino-5-oxo-4,4- diphenylimidazolidin-1- yl)methyl]benzamide | 534.2 |
| 75 | 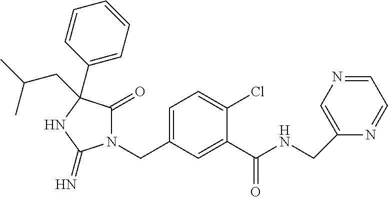 [SMILES: CC(C)CC1(c2ccccc2)NC(=N)N(Cc2ccc(Cl)c(C(=O)NCc3cnccn3)c2)C1=O] | 2-chloro-5-{[2-imino-4-(2- methylpropyl)-5-oxo-4- phenylimidazolidin-1-yl]methyl}- N-(pyrazin-2-ylmethyl)benzamide | 491.2 |
| 76 | 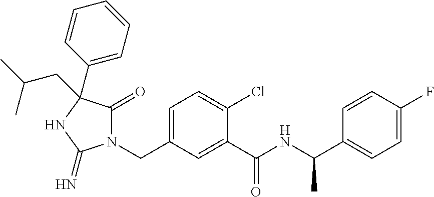 [SMILES: CC(C)CC1(c2ccccc2)NC(=N)N(Cc2ccc(Cl)c(C(=O)N[C@H](C)c3ccc(F)cc3)c2)C1=O] | 2-chloro-N-[(1R)-1-(4- fluorophenyl)ethyl]-5-{[2-imino-4- (2-methylpropyl)-5-oxo-4- phenylimidazolidin-1- yl]methyl}benzamide | 521.2 |
| 77 | 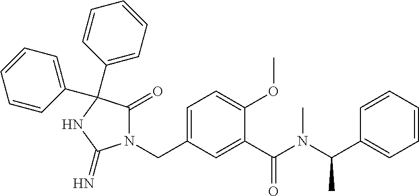 [SMILES: COc1ccc(CN2C(=N)NC(c3ccccc3)(c3ccccc3)C2=O)cc1C(=O)N(C)[C@H](C)c1ccccc1] | 5-[(2-imino-5-oxo-4,4- diphenylimidazolidin-1-yl)methyl]- 2-methoxy-N-methyl-N-[(1R)-1- phenylethyl]benzamide | 533.3 |
| 78 | 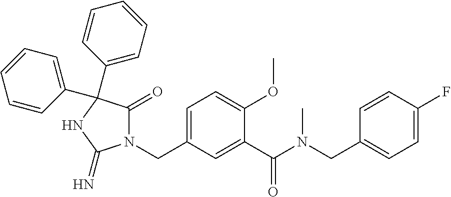 [SMILES: COc1ccc(CN2C(=N)NC(c3ccccc3)(c3ccccc3)C2=O)cc1C(=O)N(C)Cc1ccc(F)cc1] | N-(4-fluorobenzyl)-5-[(2-imino-5- oxo-4,4-diphenylimidazolidin-1- yl)methyl]-2-methoxy-N- methylbenzamide | 537.2 |
| 79 | 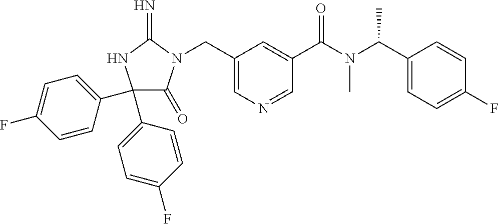 [SMILES: C[C@H](c1ccc(F)cc1)N(C)C(=O)c1cncc(CN2C(=N)NC(c3ccc(F)cc3)(c3ccc(F)cc3)C2=O)c1] | 5-{[4,4-bis(4-fluorophenyl)-2- imino-5-oxoimidazolidin-1- yl]methyl}-N-[(1R)-1-(4- fluorophenyl)ethyl]-N- methylpyridine-3-carboxamide | 558.2 |
| 80 | 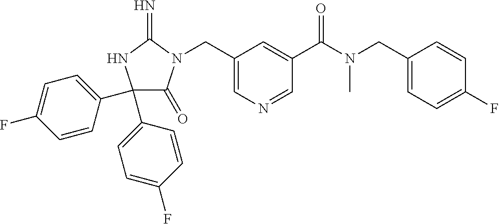 [SMILES: CN(Cc1ccc(F)cc1)C(=O)c1cncc(CN2C(=N)NC(c3ccc(F)cc3)(c3ccc(F)cc3)C2=O)c1] | 5-{[4,4-bis(4-fluorophenyl)-2- imino-5-oxoimidazolidin-1- yl]methyl}-N-(4-fluorobenzyl)-N- methylpyridine-3-carboxamide | 544.2 |
| 81 | 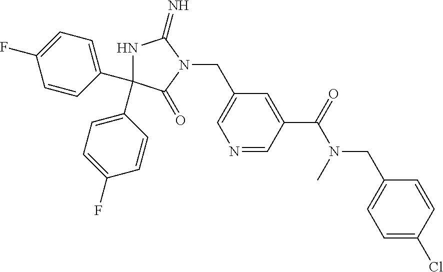 [SMILES: CN(Cc1ccc(Cl)cc1)C(=O)c1cncc(CN2C(=N)NC(c3ccc(F)cc3)(c3ccc(F)cc3)C2=O)c1] | 5-{[4,4-bis(4-fluorophenyl)-2- imino-5-oxoimidazolidin-1- yl]methyl}-N-(4-chlorobenzyl)-N- methylpyridine-3-carboxamide | 560.2 |
| 82 | 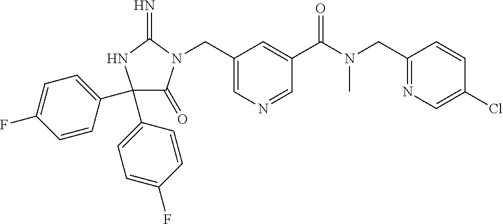 [SMILES: CN(Cc1ccc(Cl)cn1)C(=O)c1cncc(CN2C(=N)NC(c3ccc(F)cc3)(c3ccc(F)cc3)C2=O)c1] | 5-{[4,4-bis(4-fluorophenyl)-2- imino-5-oxoimidazolidin-1- yl]methyl}-N-[(5-chloropyridin-2- yl)methyl]-N-methylpyridine-3- carboxamide | 561.2 |
| 83 | 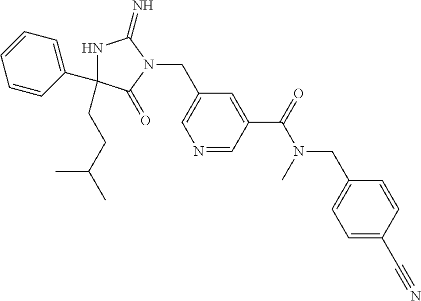 [SMILES: CC(C)CCC1(c2ccccc2)NC(=N)N(Cc2cncc(C(=O)N(C)Cc3ccc(C#N)cc3)c2)C1=O] | N-(4-cyanobenzyl)-5-{[2-imino-4- (3-methylbutyl)-5-oxo-4- phenylimidazolidin-1-yl]methyl}- N-methylpyridine-3-carboxamide | 509.3 |
| 84 | 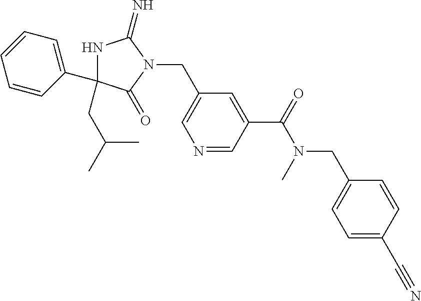 [SMILES: CC(C)CC1(c2ccccc2)NC(=N)N(Cc2cncc(C(=O)N(C)Cc3ccc(C#N)cc3)c2)C1=O] | N-(4-cyanobenzyl)-5-{[2-imino-4- (2-methylpropyl)-5-oxo-4- phenylimidazolidin-1-yl]methyl}- N-methylpyridine-3-carboxamide | 495.3 |
| 85 | 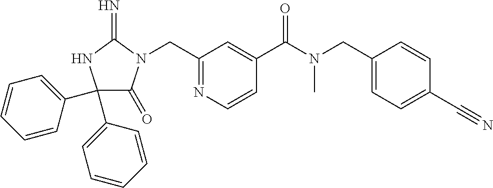 [SMILES: CN(Cc1ccc(C#N)cc1)C(=O)c1ccnc(CN2C(=N)NC(c3ccccc3)(c3ccccc3)C2=O)c1] | N-(4-cyanobenzyl)-2-[(2-imino-5- oxo-4,4-diphenylimidazolidin-1- yl)methyl]-N-methylpyridine-4- carboxamide | 515.2 |
| 86 | 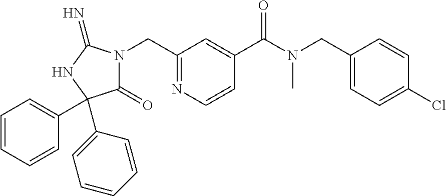 [SMILES: CN(Cc1ccc(Cl)cc1)C(=O)c1ccnc(CN2C(=N)NC(c3ccccc3)(c3ccccc3)C2=O)c1] | N-(4-chlorobenzyl)-2-[(2-imino-5- oxo-4,4-diphenylimidazolidin-1- yl)methyl]-N-methylpyridine-4- carboxamide | 524.2 |
| 87 | 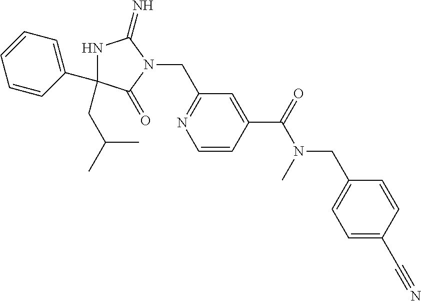 [SMILES: CC(C)CC1(c2ccccc2)NC(=N)N(Cc2cc(C(=O)N(C)Cc3ccc(C#N)cc3)ccn2)C1=O] | N-(4-cyanobenzyl)-2-{[2-imino-4- (2-methylpropyl)-5-oxo-4- phenylimidazolidin-1-yl]methyl}- N-methylpyridine-4-carboxamide | 495.3 |
| 88 | 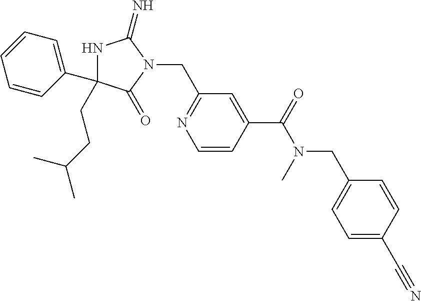 [SMILES: CC(C)CCC1(c2ccccc2)NC(=N)N(Cc2cc(C(=O)N(C)Cc3ccc(C#N)cc3)ccn2)C1=O] | N-(4-cyanobenzyl)-2-{[2-imino-4- (3-methylbutyl)-5-oxo-4- phenylimidazolidin-1-yl]methyl}- N-methylpyridine-4-carboxamide | 509.3 |
| 89 | 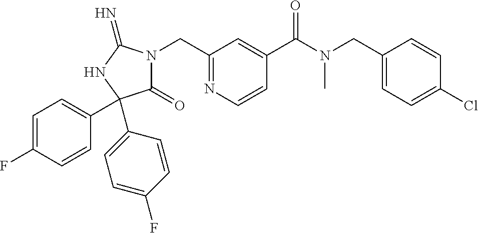 [SMILES: CN(Cc1ccc(Cl)cc1)C(=O)c1ccnc(CN2C(=N)NC(c3ccc(F)cc3)(c3ccc(F)cc3)C2=O)c1] | 2-{[4,4-bis(4-fluorophenyl)-2- imino-5-oxoimidazolidin-1- yl]methyl}-N-(4-chlorobenzyl)-N- methylpyridine-4-carboxamide | 560.2 |
| 90 | 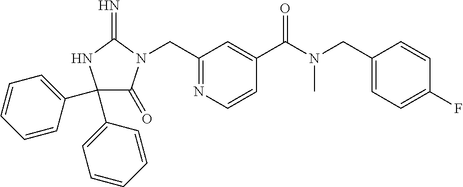 [SMILES: CN(Cc1ccc(F)cc1)C(=O)c1ccnc(CN2C(=N)NC(c3ccccc3)(c3ccccc3)C2=O)c1] | N-(4-fluorobenzyl)-2-[(2-imino-5- oxo-4,4-diphenylimidazolidin-1- yl)methyl]-N-methylpyridine-4- carboxamide | 508.2 |
| 91 | 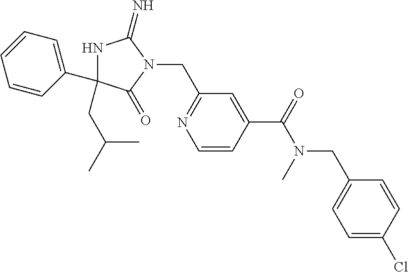 [SMILES: CC(C)CC1(c2ccccc2)NC(=N)N(Cc2cc(C(=O)N(C)Cc3ccc(Cl)cc3)ccn2)C1=O] | N-(4-chlorobenzyl)-2-{[2-imino-4- (2-methylpropyl)-5-oxo-4- phenylimidazolidin-1-yl]methyl}- N-methylpyridine-4-carboxamide | 504.2 |
| 92 | 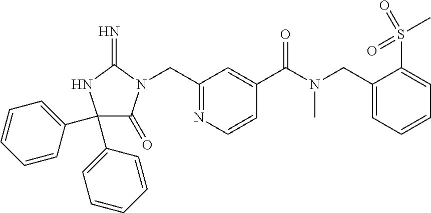 [SMILES: CN(Cc1ccccc1S(C)(=O)=O)C(=O)c1ccnc(CN2C(=N)NC(c3ccccc3)(c3ccccc3)C2=O)c1] | 2-[(2-imino-5-oxo-4,4- diphenylimidazolidin-1-yl)methyl]- N-methyl-N-[2- (methylsulfonyl)benzyl]pyridine-4- carboxamide | 568.2 |
| 93 | 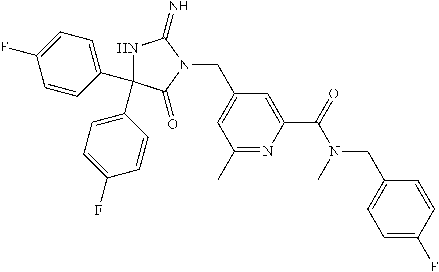 [SMILES: Cc1cc(CN2C(=N)NC(c3ccc(F)cc3)(c3ccc(F)cc3)C2=O)cc(C(=O)N(C)Cc2ccc(F)cc2)n1] | 4-{[4,4-bis(4-fluorophenyl)-2- imino-5-oxoimidazolidin-1- yl]methyl}-N-(4-fluorobenzyl)- N,6-dimethylpyridine-2- carboxamide | 558.2 |
| 94 | 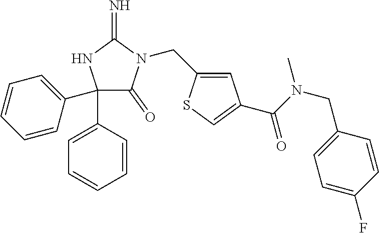 [SMILES: CN(Cc1ccc(F)cc1)C(=O)c1csc(CN2C(=N)NC(c3ccccc3)(c3ccccc3)C2=O)c1] | N-(4-fluorobenzyl)-5-[(2-imino-5- oxo-4,4-diphenylimidazolidin-1- yl)methyl]-N-methylthiophene-3- carboxamide | 513.2 |
| 95 | 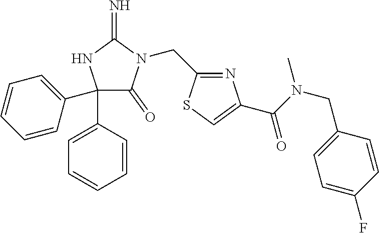 [SMILES: CN(Cc1ccc(F)cc1)C(=O)c1csc(CN2C(=N)NC(c3ccccc3)(c3ccccc3)C2=O)n1] | N-(4-fluorobenzyl)-2-[(2-imino-5- oxo-4,4-diphenylimidazolidin-1- yl)methyl]-N-methyl-1,3-thiazole- 4-carboxamide | 514.2 |

### Assay for Inhibition of Microbial Expressed HIV Protease

Inhibition of Escherichia coli expressed wild-type HIV-1 protease protein was carried out with a peptide substrate [Val-Ser-Gln-Asn-Wnaphtyl)Ala-Pro-Ile-Val]. The inhibitor compound was preincubated with HIV-1 protease enzyme in assay buffer (50 mM sodium acetate, pH 5.5, 100 mM NaCl, and 0.1% BSA) for 30 minutes at room temperature. Peptide substrate was added to 400 μM in a total volume of 20 μL containing 20 pM HIV-1 protease (final) after which the reaction was incubated for 1 hour at 30° C. The reaction was quenched by the addition of formic acid and HIV protease inhibitor indinavir to 0.012% and 150 nM final concentrations, respectively. Product formation was determined after separation of product and substrate on a ZORBAX Eclipse XDB-C18 column (Aligent Technologies, Santa Clara, Calif., USA) connected to an API 4000™ mass spectrometer (AB Sciex, Pte. Ltd., Concord Ontario, Canada) with multiple reaction monitoring (transitions were 644.5/428.9 and 615.4/422.2 (M1/M3) for product and indinavir respectively). The extent of inhibition of the reaction was determined from the peak area of the products. Analysis of the products, independently synthesized, provided quantitation standards and confirmation of the product composition. Representative compounds of the present invention exhibit inhibition of HIV-1 protease in this assay. Ic50's refer to the 50% inhibition of the cleavage of a peptide substrate by hiv protease.

Table 2 shows data obtained from the above described assays for the Compounds herein. Data shown in the table reflects the mean of at least two independent experiments.

TABLE 2

| Example Number | HIV Protease IC50 (nM) |
|---|---|
| 1 | 16.03 |
| 2 | 18.19 |
| 3 | 22.28 |
| 4 | 30.91 |
| 5 | 42.95 |
| 6 | 46.38 |
| 7 | 52.37 |
| 8 | 54.61 |
| 9 | 68.40 |
| 10 | 91.21 |
| 11 | 92.11 |
| 12 | 94.07 |
| 13 | 6.69 |
| 14 | 7.35 |
| 15 | 9.11 |
| 16 | 9.12 |
| 17 | 10.28 |
| 18 | 10.35 |
| 19 | 11.55 |
| 20 | 11.75 |
| 21 | 12.72 |
| 22 | 12.89 |
| 23 | 12.94 |
| 24 | 13.17 |
| 25 | 13.77 |
| 26 | 13.90 |
| 27 | 16.89 |
| 28 | 17.41 |
| 29 | 17.75 |
| 30 | 17.88 |
| 31 | 19.00 |
| 32 | 19.30 |
| 33 | 19.78 |
| 34 | 19.91 |
| 35 | 20.50 |
| 36 | 21.70 |
| 37 | 23.51 |
| 38 | 23.82 |
| 39 | 24.75 |
| 40 | 25.89 |
| 41 | 26.51 |
| 42 | 28.58 |
| 43 | 28.66 |
| 44 | 29.25 |
| 45 | 30.32 |
| 46 | 31.07 |
| 47 | 33.26 |
| 48 | 39.62 |
| 49 | 43.47 |
| 50 | 45.07 |
| 51 | 45.51 |
| 52 | 48.32 |
| 53 | 48.65 |
| 54 | 52.17 |
| 55 | 52.92 |
| 56 | 56.29 |
| 57 | 58.88 |
| 58 | 63.42 |
| 59 | 65.89 |
| 60 | 69.19 |
| 61 | 76.75 |
| 62 | 77.34 |
| 63 | 83.38 |
| 64 | 86.89 |
| 65 | 89.88 |
| 66 | 90.49 |
| 67 | 95.19 |
| 68 | 96.59 |
| 69 | 97.08 |
| 70 | 99.13 |
| 71 | 8.19 |
| 72 | 9.50 |
| 73 | 11.02 |
| 74 | 11.43 |
| 75 | 31.34 |
| 76 | 40.14 |
| 77 | 43.35 |
| 78 | 53.65 |
| 79 | 13.70 |
| 80 | 15.40 |
| 81 | 22.73 |
| 82 | 43.69 |
| 83 | 50.38 |
| 84 | 92.98 |
| 85 | 5.40 |
| 86 | 7.72 |
| 87 | 15.47 |
| 88 | 17.06 |
| 89 | 18.93 |
| 90 | 24.58 |
| 91 | 34.09 |
| 92 | 53.05 |
| 93 | 32.58 |
| 94 | 8.90 |
| 95 | 29.95 |

While the foregoing specification teaches the principles of the present invention, with examples provided for the purpose of illustration, the practice of the invention encompasses all of the usual variations, adaptations and/or modifications that come within the scope of the following claims.

What is claimed is:

1. A compound of structural Formula I: 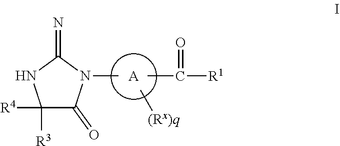
or a pharmaceutically acceptable salt thereof, wherein:
A is selected from the group consisting of (CHR²)ₚC₆₋₁₀aryl and (CHR²)ₚC₃₋₁₁ heteroaryl;
R is selected from the group consisting of hydrogen and C₁₋₆alkyl;
R^{X} is selected from the group consisting of C₁₋₆alkyl, C₁₋₃haloalkyl, halogen, SO₂C₁₋₆alkyl, and OC₁₋₆alkyl;
R¹ is selected from the group consisting of NR(CHR)ₙC₄₋₁₂heterocyclyl, and NR(CHR)ₙC₆₋₁₀aryl, wherein said aryl, and heterocyclyl is selected from substituted phenyl, pyridyl, pyrimidinyl, and pyrazinyl;
R² is selected from the group consisting of hydrogen, C₁₋₆alkyl, C₁₋₃haloalkyl, halogen, SO₂C₁₋₆alkyl, (CH₂)ₙOC₁₋₆alkyl, (CH₂)ₙC₃₋₆cycloalkyl, (CHR)ₙC₆₋₁₀aryl, (CHR)ₙC₅₋₁₀heteroaryl said alkyl, aryl, and heteroaryl optionally substituted with 1 to 3 groups of R^{d};
R³ and R⁴ are independently selected from the group consisting of C₁₋₆alkyl, (CH₂)ₙC₁₋₃ haloalkyl, (CR₂)ₙC₃₋₆cycloalkyl, (CH₂)ₙC₆₋₁₀aryl, (CH₂)ₙC₅₋₁₀heterocyclyl; said alkyl, aryl, and heterocyclyl optionally substituted with 1 to 3 groups of R^{d};
R⁵ is selected from the group consisting of hydrogen, C₁₋₆alkyl, C(O)OR, C₃₋₆cycloalkyl, SO₂R, O(CH₂)ₙC₆₋₁₀aryl, and (CH₂)ₙC₆₋₁₀aryl;
R^{d} is selected from the group consisting of C₁₋₆alkyl, C₁₋₃haloalkyl, OC₁₋₃haloalkyl, OC₁₋₆alkyl, CN, ═O, SO₂R, C(O)NR₂, C₅₋₁₀heteroaryl, C₆₋₁₀aryl, and halogen, said heteroaryl, alkyl and aryl optionally substituted with 1 to 3 groups of halogen and CN;
n is 0, 1, 2, 3, or 4;
p is 0 or 1; and
q is 0 or 1.

2. The compound according to claim 1, or a pharmaceutically acceptable salt thereof, wherein A is (CHR²)ₚC₆₋₁₀aryl.

3. The compound according to claim 1, or a pharmaceutically acceptable salt thereof, wherein the aryl is selected from the group consisting of phenyl, tetrahydronaphthalenyl, dihydroindenyl, and tetrahydrobenzoannulenyl.

4. The compound according to claim 1, or a pharmaceutically acceptable salt thereof, wherein A is (CHR²)ₚC₅₋₁₁heteroaryl, wherein the heteroaryl is selected from the group consisting of pyridyl, thiazolyl, thiophenyl, dihydrochromenyl, and dihydrothiochromenyl.

5. The compound according to claim 1, or a pharmaceutically acceptable salt thereof, wherein R¹ is substituted NR(CHR)ₙC₆₋₁₀aryl and the aryl is substituted phenyl.

6. The compound according to claim 1, or a pharmaceutically acceptable salt thereof, wherein R¹ is substituted NR(CHR)ₙC₄₋₁₂heterocyclyl and the heterocyclyl is selected from the group consisting of substituted pyridyl, pyrimidinyl, and pyrazinyl.

7. The compound according to claim 1, or a pharmaceutically acceptable salt thereof, wherein R² is hydrogen.

8. The compound according to claim 1, or a pharmaceutically acceptable salt thereof, wherein R² is selected from the group consisting of CH₃, CH₂CH₃, (CH₂)ₙCH(CH₃)₂, and (CH₂)ₙOCH(CH₃)₂ , cyclopropyl, cyclobutyl, cyclopentyl, (CHR)ₙC₆₋₁₀phenyl, and (CHR)ₙC₅₋₁₀heteroaryl

9. The compound according to claim 1, or a pharmaceutically acceptable salt thereof, wherein R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}.

10. The compound according to claim 1, or a pharmaceutically acceptable salt thereof, wherein the compounds of formula I are represented by structural formula II, or a pharmaceutically acceptable salt thereof, 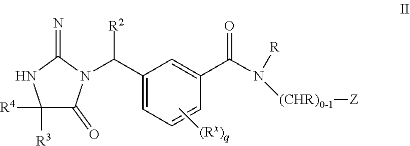
wherein R², R³, R⁴, and R^{X} are as originally described and Z is from the group consisting of substituted, phenyl, pyridyl, pyrimidinyl, and pyrazinyl.

11. The compound according to claim 10, or a pharmaceutically acceptable salt thereof, wherein Z is substituted phenyl and R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}.

12. The compound according to claim 10, or a pharmaceutically acceptable salt thereof, wherein Z is substituted pyridyl, pyrimidinyl, and pyrazinyl and R³ and R⁴ are independently selected from the group consisting of isobutyl, isopentyl, (CH₂)ₙCF₃, (CH₂)ₙcyclopropyl, phenyl, pyridyl, pyranyl, (CH₂)ₙtetrahydropyranyl, and (CH₂)ₙtetrahydrofuranyl, said isobutyl, isopentyl, cyclopropyl, phenyl, pyridyl, pyranyl, tetrahydropyranyl, and tetrahydrofuranyl optionally substituted with 1 to 3 groups of R^{d}.

13. The compound of claim 1 which is:
N-(4-chlorobenzyl)-3-{[4-(4-fluorophenyl)-2-imino-4-(3-methylbutyl)-5-oxoimidazolidin-1-yl]methyl}-N-methylbenzamide,
3-[(2-imino-5-oxo-4,4-diphenylimidazolidin-1-yl)methyl]-N-methyl-N-[2-(methylsulfonyl)-benzyl]benzamide,
N-[(1R)-1-(4-fluorophenyl)ethyl]-3-{[2-imino-4-(3-methylbutyl)-5-oxo-4-phenyllimidazolidin-1-yl]methyl}-N-methylbenzamide,
N-(4-chlorobenzyl)-3-{[2-imino-4-(2-methylpropyl)-5-oxo-4-phenylimidazolidin-1-yl]methyl}-N-methylbenzamide,
N-(4-chlorobenzyl)-3-{[2-imino-4-(3-methylbutyl)-5-oxo-4-phenylimidazolidin -1-yl]methyl}-N-methylbenzamide,
3-{[4-(3-furan-2-ylphenyl)-2-imino-5-oxo-4-phenylimidazolidin-1-yl]methyl}-N,N-dimethylbenzamide,
3-{[4,4-bis(4-fluorophenyl)-2-imino-5-oxoimidazolidin-1-yl]methyl}-N-methyl-N-(pyridin-3-ylmethyl)benzamide,
3-{[4-(3′-cyanobiphenyl-3-yl)-4-(4-fluorophenyl)-2-imino-5-oxoimidazolidin-1-yl]methyl}-N,N-dimethylbenzamide,
3-{[4,4-bis(4-fluorophenyl)-2-imino-5-oxoimidazolidin-1-yl]methyl}-N-methyl-N-(pyridin-2-ylmethyl)benzamide,
3-{[4,4-bis(4-fluorophenyl)-2-imino-5-oxoimidazolidin-1-yl]methyl}-N-methyl-N-[3-(methylsulfonyl)benzyl]benzamide,
3-{[4,4-bis(4-fluorophenyl)-2-imino-5-oxoimidazolidin-1-yl]methyl}-N-(2-methoxybenzyl)-N-methylbenzamide,
3-{[4-(4-bromophenyl)-2-imino-5-oxo-4-phenylimidazolidin-1-yl]methyl}-N,N-dimethylbenzamide,
3-[(4-biphenyl-3-yl-2-imino-5-oxo-4-phenylimidazolidin-1-yl)methyl]-N,N-dimethylbenzamide,
N-(4-chlorobenzyl)-3-{[4-(3′-cyanobiphenyl-3-yl)-4-(4-fluorophenyl)-2-imino-5-oxoimidazolidin-1-yl]methyl}-N-methylbenzamide,
3-{[4,4-bis(4-fluorophenyl)-2-imino-5-oxoimidazolidin-1-yl]methyl}-N-(2-fluorobenzyl)-N-methylbenzamide,
3-{[4,4-bis(4-fluorophenyl)-2-imino-5-oxoimidazolidin-1-yl]methyl}-N-(2-chlorobenzyl)-N-methylbenzamide,
3-{[4,4-bis(4-fluorophenyl)-2-imino-5-oxoimidazolidin-1-yl]methyl}-N-(4-cyanobenzyl)benzamide,
3-{[4,4-bis(4-fluorophenyl)-2-imino-5-oxoimidazolidin-1-yl]methyl}-N-(3-fluorobenzyl)-N-methylbenzamide,
N-(4-cyanobenzyl)-3-{[4-(3-fluorophenyl)-2-imino-4-(2-methylpropyl)-5-oxoimidazolidin-1-yl]methyl}-N-methylbenzamide,
N-(4-fluorobenzyl)-3-{(1R)-1-[2-imino-4,4-bis(2-methylpropyl)-5-oxoimidazolidin-1-yl]ethyl}-N-methylbenzamide,
2-chloro-N-(4-cyanobenzyl)-5-{[2-imino-4-(2-methylpropyl)-5-oxo-4-phenylimidazolidin-1-yl]methyl}benzamide,
2-chloro-N-(4-fluorobenzyl)-5-{[2-imino-4-(2-methylpropyl)-5-oxo-4-phenylimidazolidin-1-yl]methyl}benzamide,
2-chloro-5-[(2-imino-5-oxo-4,4-diphenylimidazolidin-1-yl)methyl]-N-(pyrazin-2-ylmethyl)benzamide,
2-chloro-N-(4-cyanobenzyl)-5-[(2-imino-5-oxo-4,4-diphenylimidazolidin-1-yl)methyl]benzamide,
2-chloro-N-[(1R)-1-(4-fluorophenyl)ethyl]-5-{[2-imino-4-(2-methylpropyl)-5-oxo-4-phenylimidazolidin-1-yl]methyl}benzamide,
5-[(2-imino-5-oxo-4,4-diphenylimidazolidin-1-yl)methyl]-2-methoxy-N-methyl-N-methyl-N-[(1R)-1-phenylethyl]benzamide,
N-(4-fluorobenzyl)-5-[(2-imino-5-oxo-4,4-diphenylimidazolidin-1-yl)methyl]-2-methoxy-N-methylbenzamide,
5-{[4,4-bis(4-fluorophenyl)-2-imino-5-oxoimidazolidin-1-yl]methyl}-N-[(1R)-1-(4-fluorophenyl)ethyl]-N-methylpyridine-3-carboxamide,
5-{[4,4-bis(4-fluorophenyl)-2-imino-5-oxoimidazolidin-1-yl]methyl}-N-(4-fluorobenzyl)-N-methylpyridine-3-carboxamide,
5-{[4,4-bis(4-fluorophenyl)-2-imino-5-oxoimidazolidin-1-yl]methyl}-N-(4-chlorobenzyl)-N-methylpyridine-3-carboxamide,
5-{[4,4-bis(4-fluorophenyl)-2-imino-5-oxoimidazolidin-1-yl]methyl}-N-[(5-chloropyridin-2-yl)methyl]-N-methylpyridine-3-carboxamide,
N-(4-cyanobenzyl)-5-{[2-imino-4-(3-methylbutyl)-5-oxo-4-phenylimidazolidin-1-yl]methyl}-N-methylpyridine-3-carboxamide,
N-(4-cyanobenzyl)-5-{[2-imino-4-(2-methylpropyl)-5-oxo-4-phenylimidazolidin-1-yl]methyl}-N-methylpyridine-3-carboxamide,
N-(4-cyanobenzyl)-2-[(2-imino-5-oxo-4,4-diphenylimidazolidin-1-yl)methyl]-N-methylpyridine-4-carboxamide,
N-(4-chlorobenzyl)-2-[(2-imino-5-oxo-4,4-diphenylimidazolidin-1-yl)methyl]-N-methylpyridine-4-carboxamide,
N-(4-cyanobenzyl)-2-{[2-imino-4-(2-methylpropyl)-5-oxo-4-phenylimidazolidin-1-yl]methyl}-N-methylpyridine-4-carboxamide,
N-(4-cyanobenzyl)-2-{[2-imino-4-(3-methylbutyl)-5-oxo-4-phenylimidazolidin-1-yl]methyl}-N-methylpyridine-4-carboxamide,
2-{[4,4-bis(4-fluorophenyl)-2-imino-5-oxoimidazolidin-1-yl]methyl}-N-(4-chlorobenzyl)-N-methylpyridine-4-carboxamide,
N-(4-fluorobenzyl)-2-[(2-imino-5-oxo-4,4-diphenylimidazolidin-1-yl)methyl]-N-methylpyridine-4-carboxamide,
N-(4-chlorobenzyl)-2-{[2-imino-4-(2-methylpropyl)-5-oxo-4-phenylimidazolidin-1-yl]methyl}-N-methylpyridine-4-carboxamide,
2-[(2-imino-5-oxo-4,4-diphenylimidazolidin-1-yl)methyl]-N-methyl-N-[2-(methylsulfonyl)benzyl]pyridine-4-carboxamide,
4-{[4,4-bis(4-fluorophenyl)-2-imino-5-oxoimidazolidin-1-yl]methyl}-N-(4-fluorobenzyl)-N,6-dimethylpyridine-2-carboxamide,
N-(4-fluorobenzyl)-5-[(2-imino-5-oxo-4,4-diphenylimidazolidin-1-yl)methyl]-N-methylthiophene-3-carboxamide,
N-(4-fluorobenzyl)-2-[(2-imino-5-oxo-4,4-diphenylimidazolidin-1-yl)methyl]-N-methyl-1,3-thiazole-4-carboxamide, or a pharmaceutically acceptable salt thereof.

14. A pharmaceutical composition comprising an effective amount of a compound according to claim 1, or a pharmaceutically acceptable salt thereof, and a pharmaceutically acceptable carrier.

15. A method for the treatment or prophylaxis of infection by HIV or for the treatment, prophylaxis, or delay in the onset of AIDS in a subject in need thereof, which comprises administering to the subject an effective amount of the compound according to claim 1, or a pharmaceutically acceptable salt thereof.

16. A compound of any one of claim 1, or a pharmaceutically acceptable salt thereof, for use in the preparation of a medicament for the inhibition of HIV protease, for the treatment or prophylaxis of infection by HIV, or for the treatment, prophylaxis, or delay in the onset of AIDS in a subject in need thereof.

17. A pharmaceutical composition comprising an effective amount of a compound of claim 1, or a pharmaceutically acceptable salt thereof, and a pharmaceutically acceptable carrier and further comprising an effective amount of an anti-HIV agent selected from the group consisting of HIV antiviral agents, immunomodulators, and anti-infective agents.

18. The pharmaceutical composition of claim 17, wherein the anti-HIV agent is an antiviral selected from the group consisting of HIV protease inhibitors, HIV reverse transcriptase inhibitors, HIV integrase inhibitors, HIV fusion inhibitors, HIV entry inhibitors, and HIV maturation inhibitors.

19. A compound of claim 1 for use in therapy.
